(19) United States
(12) Patent Application Publication
(10) Pub. No.: US 2005/0129732 A1
(43) Pub. Date: Jun. 16, 2005

(54) Biodegradable, antibiotic, controlled release tape

(75) Inventor: Reid M. Rubsamen, Alamo, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: Flow Focusing, Inc.

(21) Appl. No.: 11/004,050

(22) Filed: Dec. 2, 2004

Related U.S. Application Data
(63) Continuation-in-part of application No. 10/618,255, filed on Jul. 10, 2003.
Application No. 10/618,255 is a continuation-in-part of application No. 10/195,046, filed on Jul. 12, 2002.
(60) Provisional application No. 60/326,675, filed on Oct. 2, 2001.
(60) Provisional application No. 60/305,364, filed on Jul. 13, 2001.
(60) Provisional application No. 60/526,925, filed on Dec. 3, 2003.
(60) Provisional application No. 60/536,019, filed on Jan. 12, 2004.

(51) Int. Cl. A61K 9/70; A61F 13/00; A61K 9/14

(52) U.S. Cl. 424/423; 424/443; 428/40.2

(57) ABSTRACT

A biodegradable tape with antibiotic properties is disclosed. The tape may be used in a method of the invention to wrap a surgical implant such as a surgical screw thereby providing a tighter fit and antibiotic effects on the surrounding environment. The tape is produced in a range of widths, lengths and thicknesses to meet particular needs. The tape may be produced in two or more layers which layers can entrap and provide for controlled release of an antibiotic.

### CROSS-REFERENCE

This application is a continuation-in-part of U.S. application Ser. No. 10/618,255 filed Jul. 10, 2003 which is a continuation-in-part of application Ser. No. 10/195,046 filed Jul. 12, 2002 which claims the benefit of 60/326,675 filed Oct. 2, 2001 and 60/305,364 filed Jul. 13, 2001. Further this application claims the benefit of U.S. Provisional Application No. 60/526,925, filed Dec. 3, 2003 and 60/536,019 filed Jan. 12, 2004 all of which applications are incorporated herein by reference.

### FIELD OF THE INVENTION

The invention relates generally to a surgical device and particularly to a biodegradable tape comprising an antibiotic, which tape is used to wrap a surgical implant such as a bar, plate and/or screw in order to inhibit infection.

### BACKGROUND OF THE INVENTION

In order to improve the effectiveness and functionality of wound dressings and surgical implants, various attempts have been made to incorporate them with a variety of medicaments such as antibiotics, analgesics, and the like—see U.S. Pat. No. 5,972,366.

Examples of antibacterial wound dressings are disclosed in U.S. Pat. No. 4,191,743 to Klemm et al., U.S. Pat. No. 2,804,424 to Stirn et al., and U.S. Pat. No. 2,809,149 to Cusumano. Similarly, U.S. Pat. No. 3,987,797 to Stephenson discloses a suture rendered antimicrobial.

Dressings which attempt to promote wound healing are disclosed in U.S. Pat. No. 5,124,155 to Reich. Many prior art surgical bandages and dressings which incorporate medications are made by soaking the material in an aqueous solution of the medicine. This can render the carrier brittle and inflexible upon drying. Moreover, it is difficult to control the rate of release of the medicament, or its effect on peripheral tissues, when it is applied to the carrier dissolved in a liquid state. Also, many important medicines are water insoluble and cannot be applied by this technique. Alternatively, the medicament is applied to the dressing or implant as a powder or dust which is quickly released and possesses a danger that large drug particles may irritate tissue or enter the circulatory system where they can block capillaries.

In addition to externally applied dressings, it is also known to impregnate an implantable surgical material with a medicament. For example, U.S. Pat. No. 5,197,977 to Hoffman Jr. et al. disclose a synthetic vascular graft that is impregnated with collagen and a medicament.

Additionally, Boyes-Varley et al. in Int. J. and Maxillafac. Surg. 1988; 17:138-141, describe the use in an animal study of a the Gelfoam.RTM. brand sponge with a saline solution of medicaments. However, the Physicians' Desk Reference, (Medical Economics, Co., Oradell, N.J.) 1992 edition warns that “it is not recommended that Gelfoam.RTM. be saturated with an antibiotic solution or dusted with antibiotic powder.” A similar warning is provided with the entry of another popular surgical implant—the Surgicel.RTM. brand absorbable hemostat—which states that “the Surgicel.RTM. hemostat should not be impregnated with anti-infective agents.”

It would be desirable to have a method for safely and effectively impregnating externally applied dressings as well as implantable sponges and hemostats, especially the popular Gelfoam.RTM. and Surgicel.RTM. brands. More particularly, it would be desirable to impregnate the dressings or implants which may be metal such as metal screws with medicament in neither a solute nor a powder form, but a form which permits the drug concentration and release rate to be controlled.

### SUMMARY OF THE INVENTION

A biodegradable tape with antibiotic properties is disclosed. The tape may be used in a method of the invention to wrap a surgical implant such as a surgical screw thereby providing a tighter fit in bone and provide antibiotic effects on the surrounding environment. The tape is produced in a range of widths, lengths and thicknesses to meet particular needs. The tape may be produced in two or more layers which layers can entrap and provide for controlled release of an antibiotic. The antibiotic may be in a pure form, mixed with excipients, or being in the form of coated particles to provide for controlled release of antibiotic over a desired period.

An aspect of the invention is a biodegradable, controlled release antibiotic tape and the tape may be comprised of a polymer which may be a synthetic polymer (e.g. poly(vinyl alcohol) or a natural polymer (e.g. collagen).

Another aspect of the invention is a method whereby an antibiotic biodegradable, controlled release tape is used to wrap a surgical implant which is then placed in a patient.

Yet another aspect of the invention is a method of producing an antibiotic, biodegradable tape whereby polymeric strips are layered together entrapping an antibiotic therein and providing a controlled release result.

In an aspect of the invention small holes which may be hemispherical in shape are formed in the implant such as a screw and the holes are filled with controlled release antibiotic particles.

In another embodiment screws or other implants with the antibiotic filled holes are wrapped with a biodegradable, antibiotic tape of the invention.

In another embodiment the tape of the invention provides an initial quick release or controlled release of the antibiotic followed by controlled release from the antibiotic filling the holes in screw or other implant.

Another object of the invention is to change the standard of care in surgery so that implants such as screws, braces and other metal implants are wrapped with a tape of the invention to thereby decrease the occurrence of infection.

Gentamycin sulphate (GS) is an aminoglycoside antibiotic. This antimicrobial agent has many properties making it a good candidate for local administration including, tissue compatibility, bactericidal activity, low rate of bactericidal resistance, broad spectrum activity, low resorption rate, and lack of interference with wound healing and high degree of chemical stablity in presence of biological material such as fibrin.

The most frequently used biodegradable polymers are polyesters poly(lactide acid) (PLA) and poly(D,L-lactide-co-glycolide) (PLGA), as they are well tolerated and degrade to toxicologically harmless products. As such, these polymers have been commonly used in the development of implantable drug delivery systems.

Traditional drug micro encapsulation methods such as conventional spray-drying or emulsion-evaporation methods do not provide uniformly sized particles with the desired drug content.

It is an object of this invention to demonstrate that the Flow Focusing process can be utilized to fabricate precisely sized microencapsulated spheres of PLGA and GS.

It is another object of this invention to demonstrate that the in-vitro release profile from the GS/PLGA microencapsulated spheres is related to the particle size of the spheres.

It is another object of this invention to describe the novel application of combining the different sizes of GS/PLGA microencapsulated spheres in proportions to produce a desired net total release profile of GS.

It is another object if this invention to describe the application of prevention of surgical site infection in patients being treated with implantation hardware by coating this hardware with a pre-determined combination of sizes of microencapsulated GS/PLGA spheres.

It is another object of this invention to describe vehicles in which the microencapsulated GS/PLGA micro spheres can be contained prior to application to implanted hardware such as biodegradable tape or a foam emulsion.

These and other objects, advantages, and features of the invention will become apparent to those persons skilled in the art upon reading the details of the various embodiments as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 is a schematic view of a spray drying device.

FIG. 2 is a schematic view of an embodiment of an extrusion device used to create spherical particles.

FIG. 3 is a schematic view of an embodiment of an extrusion device used to create spherical coated particles.

FIG. 4 is a graph of time versus (amount of a compound dissolved minus the amount eliminated) for a single particle or group to substantially identical particles.

FIG. 5 is a graph of time versus (amount of a compound dissolved minus the amount eliminated) for two different particles or two different groups of particles where the particles within a given group are substantially identical and also showing the combined effect of the two groups.

FIG. 6 is a graph of time versus (amount of a compound dissolved minus the amount eliminated) for three different particles or three different groups of particles where the particles within a given group are substantially identical and also showing the combined effect of the three groups.

FIG. 7 is a perspective view of a surgical screw with indentations around its surface.

FIG. 8 is the screw of FIG. 7 with controlled release spheres in the indentations.

FIG. 9 is a perspective view of a surgical screw with indentations only on the upper, non-leading edges of the screw ridges.

FIG. 10 is the screw of FIG. 9 with controlled release spheres in the indentations.

FIG. 11 shows a finger held tape holding device of the invention with a tape in place.

FIG. 12 shows a hand held tape holding device with a tape in place.

FIG. 13 shows the tape of the device of FIG. 11 being placed on an implant screw.

FIG. 14 shows a hand held tape holding device of the invention with the tape being wrapped around a screw as shown in FIG. 8.

FIG. 15 is a schematic diagram of what is referred to as a “flow focusing” type nozzle and an associated liquid and gas input along with the schematic representation of a drying chamber into which the particles flow.

FIG. 16 is a schematic diagram of a flow focusing nozzle wherein D₀=D=H=150 microns.

FIG. 17 includes FIG. 17A which is a front view in a 6:1 scale of a flow focusing nozzle; FIG. 17B is a cross sectional view along the lines A-A of FIG. 17A also in 6:1 scale showing the nozzle with the focusing gas inlet; and FIG. 17C is a detailed 40:1 scale view of the circled portion of FIG. 17B.

FIG. 18A is a cross sectional view of a nozzle with the dimensions given in millimeters and FIG. 18B is a front view of the nozzle in a 8:1 as with FIG. 18A and also with the numbers shown in millimeters.

FIG. 19A of a nozzle at a 15:1 scale and FIG. 19B is a front view of a nozzle also at a 15:1 scale.

FIG. 20 is a graph showing the microsphere size distribution from non-dried samples.

FIG. 21 is a photograph of actual spheres produced with a flow focusing nozzle.

FIG. 22 is a graph of the microsphere size distribution of non-dried samples.

FIG. 23 is a photograph of actual microspheres produced using the invention wherein the bar shown in the photograph is 10 microns in length.

FIG. 24 is a graph of microsphere size distributions from non-dried samples.

FIG. 24 is a photograph of actual dried samples.

FIG. 25 is a photograph of actual microspheres produced in accordance with the invention wherein again the bar shown is 10 microns in length.

FIG. 26 is a Petri dish showing zones of inhibition (ZOI) as clear areas wherein the number 5 at the top indicates the use of the 7 micron spheres. The numbers 2, 4, 6 and 8 indicate the time points in hours. The lower case “f” in the center indicates the residual microspheres collected after the last time point is taken.

FIG. 27 is a photograph of an actual Petri dish as with FIG. 26. The number 6 at the top indicates the use of the eleven micron sphere size. The numbers 2, 4, 6 and 8 indicate the time points in hours. The lower case “f” in the center indicates the residual microspheres collected after the last time point was taken.

FIG. 28 is a photograph of an actual Petri dish with the ZOI shown as clear areas. The number 8 at the top indicates the use of the 23 micron sphere size. The numbers 2, 4, 6, and 8 indicate the time points in hours. The lower case “f” in the center indicates the residual microspheres collected after the last time point was taken.

FIG. 29 is a graph wherein the zones of inhibition are plotted as a function of time expressed as an aggregate amount released. The smaller microspheres are seen to release the antibiotic faster than the larger microspheres.

### DETAILED DESCRIPTION OF THE INVENTION

Before the present biodegradable, controlled release antibiotic tape and method for using such are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a layer” includes a plurality of such layers and reference to “the method” includes reference to one or more methods and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “tape” refers to a narrow strip of material. The tape has a length dimension larger than a width dimension and width dimension which is larger than its thickness dimension. The tape may be a few millimeters to 10's of meters in length and a millimeter to several centimeters in width. The tape may be comprised of a biodegradable material such as a polymer and have a thickness of from 0.01 mm to 10 mm. Typical length may be 1 cm to 10 cm, typical width may be 2 mm to about 1-2 cm, and typical thickness may be about 0.5 mm to 5 mm.

The terms “synergistic”, “synergistic effect” and like are used herein to describe improved treatment effects obtained by combining one or more active components together in a composition or in a method of treatment. Although a synergistic effect in some field is meant an effect which is more than additive (e.g., 1+1=3) in the field of treating many diseases an additive (1+1=2) or less than additive (1+1=1.6) effect may be synergistic. For example, if one active ingredient removed 50% of an infection and a second active ingredient removed 50% of the infection the combined (and merely additional) effect would be 100% removal of the infection. However, the effect of both would not be expected to remove 100% of the infection. Often, two active ingredients have no better or even worse results than either component by itself. If an additive effect could be obtained merely by combining treatments than multiple ingredients could be applied to successfully treat any disease and such is not the case.

“Osteomyelitis” is an inflammation or an infection in the bone marrow and/or surrounding bone. The disease may be classified as either acute or chronic, depending on the length of time the infection or symptoms persist. Symptoms may include pain, warmth and/or swelling in the bone. Chronic osteomyelitis may last for years, with slow death of bone tissue from a reduced blood supply. Signs and symptoms may be absent, however, causing difficulty in diagnosing the chronic infection. The invention includes treating osteomyelitis in connection with surgical implants and in particular surgical screws.

Osteomyelitis is an infection involving the bone. It often afflicts the growing individual. The bones usually affected are the weight-bearing bones before the physis has closed. Exogenous osteomyelitis occurs from trauma, sometimes as trivial as falling on a stick. Hematogenous osteomyelitis occurs from bacteria circulating in the bloodstream. Acute and chronic subtypes are classified according to the timing and duration of the infection.

Publications providing further details regarding osteomyelitis include the following:

Dirschl D R, Almekinders LC. Osteomyelitis. Drugs. 1993; 45: 29-43.

Ehara S. Complications of skeletal trauma. Radiol Clin North Am. 1997; 35: 767-781.

Sammak B, Abd El Bagi M, Al Shahed M, et al. Osteomyelitis: a review of currently used imaging techniques. Eur Radiol. 1999; 9: 894-900.

Waldvogel F, Medoff G, Swartz M. Osteomyelitis: a review of clinical features, therapeutic considerations and unusual aspects (I). N Engl J. Med. 1970; 282: 198-206.

Widmer AF. New developments in diagnosis and treatment of infection in orthopedic implants. Clin Infect Dis. 2001; 33: S94-S106.

The terms “treatment”, “treating” and the like are used herein to generally mean obtaining a desired pharmacological or physiological effect. The effect may be prophylactic in terms of completely or partially preventing a disease such as an infection or symptom thereof and may be therapeutic in terms of partially or completely curing a disease and/or adverse effect attributed to the disease or infection. “Treatment” as used herein covers any treatment of any disease and specifically infectious bacterial, fungal, parasitic, and viral infections, in a mammal, particularly a human, and includes:
(a) preventing the disease or infection from occurring in the subject which may be predisposed to the disease but has not yet been diagnosed as having it;
(b) inhibiting the disease or infection, i.e. arresting its development; or
(c) relieving the disease or infection, i.e. causing regression of the disease or infection. The infection is directed towards treating patients with wounds and in particular bone damage and is directed towards the use of surgical implants such as screws in order to prevent infection or more particularly preventing osteomyelitis. In connection with the invention treating can include surgical procedures such as the implantation of orthopedic components with antimicrobial controlled release compositions bound to the surface of the implant so as to treat (prevent) osteomyelitis. However, a tape of the invention can comprise other pharmaceutically active drugs (e.g. anticoagulants) and used in stents.

The term “Controlled Release” is used to mean any one of a number of extended release dosage forms. The following terms may be considered to be substantially equivalent to controlled release, for the purposes of the present invention: continuous release, controlled release, delayed release, depot, gradual release, long-term release, programmed release, prolonged release, proportionate release, protracted release, repository, retard, slow release, spaced release, sustained release, time coat, timed release, delayed action, extended action, layered-time action, long acting, prolonged action, repeated action, slowing acting, sustained action, sustained-action medications, and extended release. Further discussions of these terms may be found in Lesczek Krowczynski, Extended-Release Dosage Forms, 1987 (CRC Press, Inc.).

As used herein “Collagen” means pharmaceutical grade collagen used in the treatment of human patients. Collagen is a fibrous protein that form fibrils having a very high tensile strength and that has been found in most multicellular organisms. Collagen serves to hold cells and tissues together and to direct the development of mature tissue. Collagen is the major fibrous protein in skin, cartilage, bone, tendon, blood vessels and teeth.

There are many types of collagen which differ from each other to meet the requirements of various tissues. Some examples of types of collagen are as follows: type one [α1(I)]₂ α2 which is found in skin, tendon, bone and cornea; type two [α1(II)₃ which is found in cartilage intervertebral disc, and the vitreous body; type three [α]1(III)]₃ which can be found in skin and the cardiovascular system; type four [α1(IV)]₂ α2(IV) which can be found in basement membrane; type five [α1(V)]₂ α2(V) and α1(V)α2(V)α3(V) which is found in the placenta and cornea. Examples of newly identified forms of collagen include: type seven (VII) which is found in anchoring fibrils beneath many epithelial; and types nine (IX), ten (X) and eleven (XI), which are minor constituents of cartilage.

The chemical characterization of native collagen was difficult since its low solubility made isolation of collagen a tedious task. Eventually, it was discovered that collagen from tissues of young animals was not as extensively cross linked as that of mature tissues and thus was more amenable to extraction. For example, the basic structural unit of type I collagen, tropo-collagen, can be extracted in intact form from some young, collagen-containing animal tissues.

Substantial information can be found in patents and publications relating to uses of Collagen. For example, see U.S. Pat. Nos. 4,294,241; 4,668,516; 5,640,941; and 5,716,411 all of which are incorporated herein by reference as are the publications and patents cited in these patents to disclose and describe various ways of using collagen which can in turn be mixed with and administered and used with antibiotic formulations of the present invention.

### Invention in General

A biodegradable tape is disclosed. The tape is comprised of a biodegradable and biocompatible materials generally a polymer (e.g. poly(vinyl alcohol) or collagen which polymer may incorporate a pure antibiotic and/or controlled release antibiotic spheres. The tape is a thin strip of material which may have a biocompatible adhesive thereon and/or be comprised of a thin layer of stretchable polymer which may be pulled over and allowed to adhere to the surface of an implant such as a metal screw. The tape has a pharmaceutically active compound coated on it, embedded in it and/or attached to it in some manner. That compound may be an antimicrobial, antibiotic type drug used to prevent infection e.g. prevent osteomyelitis. Alternatively, the compound may be an anticoagulant used to prevent clots when the tape is used on a vascular stent. The tape may comprise combinations of two or more drugs to obtain a desired result. For example, an antibiotic and an anticoagulant may both be incorporated into or adhered to the tape.

Pathogens infect bone in posttraumatic osteomyelitis after a recent fracture. Bacteria, fungus and other microorganisms are typically the causative agents. The more susceptible a bone is to fracturing, the greater the chances of becoming infected and developing disease. Trauma from recent injuries and diabetes are major risk factors for osteomyelitis. The bone can be directly infected from the wound or indirectly via the blood from another site of infection, called hematogenous osteomyelitis. The vertebrae and pelvis are often affected in adults in this blood-borne variety, while children are usually affected in long bones.

The incidence of osteomyelitis after open fractures is reported to be 2% to 16%, depending significantly on the grade of trauma and the type of treatment administered. Prompt and thorough treatment helps reduce the risk of infection, decreasing the probability of developing osteomyelitis. This is particularly important for patients with the following risk factors: diabetes, altered immune states and recent trauma. The tibia is the most frequent site of posttraumatic osteomyelitis, since it is the most vulnerable bone with the least vigorous blood supply in the body.

The classification of osteomyelitis can be broken down into the following categories: exogenous osteomyelitis (47%), secondary to vascular insufficiency (34%) and hematogenous osteomyelitis (19%). The implantation of an orthopedic device (pins, plate, screws, artificial joint) can also seed infection as a nidus for pathogens, and therefore create post-operative osteomyelitis.

Risk factors include the growing skeleton. Any bone can be affected but it is usually the weight-bearing bones before the physis has closed. At the physis on the metaphyseal side, end arteries form a capillar loop which may rupture following minor trauma. This region of blood stasis may attract circulating bacteria and all people have bacteria circulating, periodically. Once they escaped through the vascular system, bacteria can proliferate in surrounding tissues.

The presence of bacteria alone in an open fracture is not sufficient to cause osteomyelitis. In many cases, the body's immune system is capable of preventing the colonization of pathogens. The micro-environment determines whether infection occurs. The timing and extent of treatment are critical in determining whether infection develops. The likelihood of developing osteomyelitis increases with impaired immune function, extensive tissue damage, or reduced blood supply to the affected area. Patients with diabetes, poor circulation or low white blood cell count are at greater risk. Bacteria on a surface on an implant such as a screw or on the surface of the bone where the implant or screw is placed may be isolated from circulation and form the basis of an infection.

Bacterial or fungal infection cause most osteomyelitis. Infection induces a large polymorphonuclear response from bone marrow, particularly staphylococcus aureus, streptococcus and haemophilus influenza. Staphylococcus infection predominates today and before the era of antibiotics.

The diagnosis of osteomyelitis may be made from clinical, laboratory and imaging studies. When the skeletal system is involved, pain, fever and leukocytosis (an increase in white blood cell count due to infection or inflammation) occur. The affected area is painful. Initial x-rays are typically normal. As early as 4 days, an area of lucency may be seen on x-ray.

Usually, the changes are not recognized until 10 days or two weeks have passed. Subperiosteal new bone formation in the affected area is present, representing periosteal elevation from encroaching pus. If not successfully treated, pus enlarges the bone appearing as increased lucency, which surrounds sclerotic, dead bone. This inner dead bone is called the sequestrum (sequestered from blood supply), and the outer periosteal reaction laminates to form the involucrum.

Draining sinuses develop when the pressure of pus exceeds the containment of the soft tissue. This further deprives the bone of its blood supply. This in turn harbors more bacteria, and the process cannot be reversed until extensive debridement of the area occurs-until the environment changes to one that promotes healing.

Osteomyelitis is an infection involving the bone. It often afflicts the growing individual. The bones usually affected are the weight-bearing bones before the physis has closed. Exogenous osteomyelitis occurs from trauma, sometimes as trivial as falling on a stick. Hematogenous osteomyelitis occurs from bacteria circulating in the bloodstream. Acute and chronic subtypes are classified according to the timing and duration of the infection.

### Mathmatics of Controlled Release Particles

Biodegradable polymers, and/or biodegradable adhesive incorporate drugs such as antibiotic in spherical particles. These particles are used to obtain controlled release the drug. The results may be obtained using groups of particles based on mathematics. For any given particle having a given amount of surface area the rate of dissolution will decrease as the particle dissolves and the total available surface area decreases. Thus, a spherical particle with two square units of available surface area which dissolves at a rate of X per unit of time will be dissolving at a rate of X/2 per unit of time once the particle has dissolved so that it has one square unit of available surface area. This assumes a constant environment unaffected by the dissolution and the circulatory systems of a human approximates such an environment.

By combining two different particles each comprised of the same material but of a different size the combined rate of the two particles together is different from either particle by itself. The combined rate of a small and a large particle is slower than two large particles and faster than two small particles.

A particle with a large available surface area has a more rapid dissolution rate that a particle with a small available surface area. However, assuming the same total volume in two groups of particles the group of smaller particles has a faster dissolution rate than the group of larger particles because the group of smaller particles will have a larger available surface area than the group of larger particles.

It is often desirable to deliver a predetermined amount of compound (such as a drug) to a system (such as a human) at a rate which maintains the compound in the system (or target area such as around a screw) at a desired level over a desired period of time. When the total amount (weight and volume) is fixed the rate of dissolution is dictated by the available surface area. One spherical particle with a given total volume will present approximately half the surface area as ten particles with the same combined volume as the one particle. Each time the number of particles is increased by a multiple of ten (and the combined volume remains constant) the total available surface area approximately doubles. The following provides specific examples of how the total available surface area increases as the same total volume (e.g. a drug) is included in larger numbers of spheres.

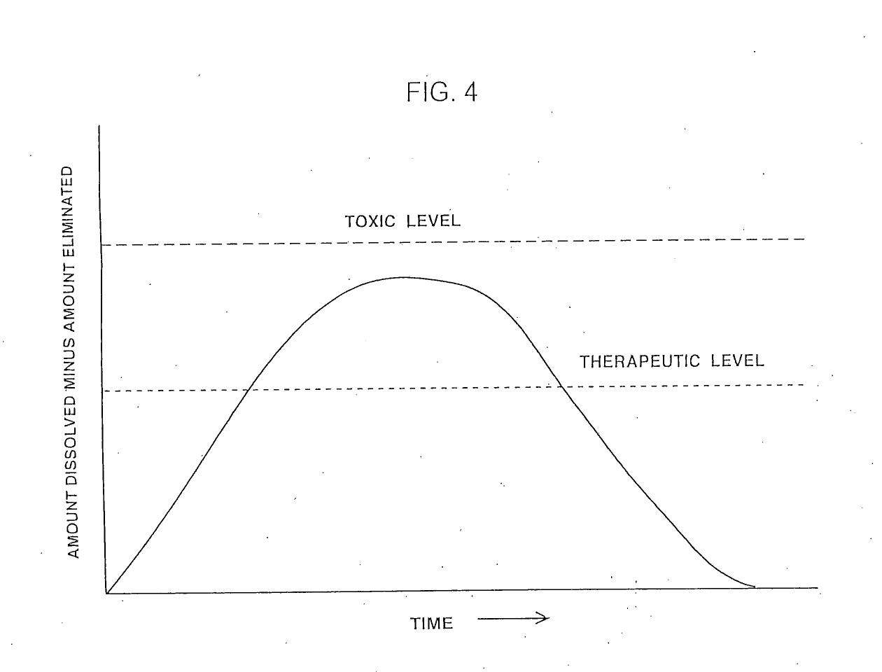

Devices of the invention may include some antimicrobial such as an antibiotic for immediate release to provide a fast antimicrobial effect in the surrounding area. Further, greater numbers of groups of different particles can increase the duration time the drug is released and decrease changes in the concentration of the drug in the surrounding areas over time. Thus, 2 or more, 3 or more, 4 or more or 5 or more groups can be used to maintain the desired therapeutic level over time—see FIGS. 4, 5 and 6.

### Specifics of Particle Sizes

Assuming a particular device or a tape used to wrap devices will be coated with particles which will contain a total volume of 2 cubic centimeters the size a single sphere which will hold a 2 cc volume can be readily calculated using the formula for the volume of a sphere as follows:
Volume of a sphere=(4/3)πr³ if the volume of a sphere is 2 cc then
2 cc=(4/3)πr³
2=(4/3) 3.14159 r³
2=4.1887867 r 3
0.477645=r³
0.781592 cm=r
r=7,815 micrometers
diameter=d=2r=15,630 micrometers

The formula for the surface area of a sphere is 4πr². Because “r” was found to be 0.781592 cm the surface area=4(3.14159)(0.781592)=9.8217 cm².

The formula for the volume of a sphere can be readily modified to determine the volume of any number of spheres “n” needed to make a total volume of 2 cubic centimeters.
2 cc=n(4/3)πr³

This formula was solved above for “n” equals “1” and can be solved for any “n.” For example, when “n” is 10 the formula becomes
2 cc=10(4/3)πr³
2 cc=10(4/3)3.14159 r³
2 cc=41.887867 r³
0.0477645=r³
0.362783 cm=r
r=3627 micrometers
d=7254 micrometers

The volume of each sphere is 0.2 cm³ and the surface area of each sphere is 1.65388 cm². Thus, the total volume of the 10 spheres remains the same (i.e. 2 cc) but the surface area of all 10 spheres is 16.5 cm² as compared to 9.8217 cm when “n” was one.

When “n” equals 100 the radius “r” can be solved for and found to be 0.1684 cm with the volume of each of the 100 spheres being 0.02 cm³. The surface area of each sphere is 0.3563 cm² and the combined surface area of all 100 spheres is 35.63 cm²—the combined volume remains the same at 2 cm³. The equations for the surface area and volume can be used to solve for the radius “r” and diameter “d” of any number of spheres “n” which equal a total volume of 2 cm³ and the results are provided below.

TABLE 1
Total volume is 2 cm³

| | | | | Surface |
|---|---|---|---|---|
| | | | Surface | Area |
| | r(micro- | | area | Volume |
| N | meters) | D | (cm²) | (cm⁻¹) |
| 1 | 7815 | 15,630 | 9.8217 | 4.91085 |
| 10 | 3627 | 7,254 | 16.5 | 8.25 |
| 100 | 1684 | 3,378 | 35.63 | 17.815 |
| 1,000 | 781 | 1,562 | 76.766 | 38.383 |
| 10,000 | 362 | 724 | 165 | 82.5 |
| 100,000 | 168 | 336 | 356 | 178 |
| 1,000,000 | 78 | 156 | 768 | 384 |
| 10,000,000 | 36 | 72 | 1,653 | 826.5 |
| 100,000,000 | 16.8 | 33.6 | 3,563 | 1781.5 |
| 1,000,000,000 | 7.8 | 15.6 | 7,677 | 3838.5 |
| 10,000,000,000 | 3.6 | 7.2 | 16,539 | 8269.5 |
| 100,000,000,000 | 1.6 | 3.2 | 35,631 | 17815.5 |

From the above it can be seen that when “n” is increased by a factor of 10 and total combined volume is maintained constant at 2.0 and the combined surface area of all of the spheres increases by approximately a factor of 2 for each increase of 10× for n.

Although the surface area approximately doubles as “n” increases by a factor of ten the absolute effect of the doubling is small when “n” is increased from 1 to 10 to 100. Specifically, the increase in surface area from 9.8 to 16.5 is only an increase of 6.7 cm² and from 16.5 to 35.6 is only an increase of 19.1 cm². However, when “n” increases from 10⁹ to 10¹⁰ the surface area increases from 7677 to 16,539 resulting in an increase of 8,862 cm². When “n” increases from 10¹⁰ to 10¹¹ the surface area increases from 16,539 to 35,631 resulting in an increase of 18,992 cm².

For “n” at the extremes of the calculations provided above the gross increase in surface area is as follows:

TABLE 2

| N | gross increase in surface area (cm²) |
|---|---|
| 1 to 10 | 6.7 |
| 10 to 100 | 19.1 |
| 10⁹ to 10¹⁰ | 8,8863 |
| 10¹⁰ to 10¹¹ | 18,992 |

The larger the available surface area the faster the rate of dissolution of the solute drug assuming the solvent is not saturated. In nearly all situations the solute drug will only be administered to the surrounding environment of the solvent (e.g. tissue such as bone) in relatively small amounts. Accordingly, the solvent never approaches saturation.

Formulations of the invention are described and claimed here and such formulations may have two, three or a plurality of different groups of particles therein. The formulation suspension may be created where a first group has a first surface area and a second group has 1,000 square centimeters or more surface area than the first group or e.g. 2,000 or more; 5,000 or more; or 10,000 or more square centimeters of surface area more than the surface area of the first group. Formulations of suspensions of particles may be created whereby a plurality of different groups are present and the total surface area of any one group different from the total surface area of any other group by a desired amount e.g. 1,000; 2,000; 3,000; 4,000; 5,000; and 10,000 or more square centimeters of surface area.

Using data such as generated in Table 1 and the results of Table 2 a formulation of the invention can be created which provides a desired release profile. The solvent is the surrounding environment which can be any area where the drug is delivered including the blood, body fluids, tissue including bone. A screw wrapped with a tape of the invention may release antibiotic to an area surrounding the screw extending outward about 1 to 2 cm or less in all directions and beyond that sphere of influence provide very small, non-therapeutic, and even undetectable levels of the antibiotic.

The solvent or surrounding environment into which the drug is administered can be assumed to be known within a given environment (e.g. bone tissue or blood) in a given species of animal (e.g. human). Thus, the unknown that remains is the rate of dissolution of a particle of known size in a given solvent. After calculating the rate of release “R” (weight or volume dissolved per unit of time) for a known particle size the rate of dissolution of other particle sizes with different available surface areas can be calculated. Assuming all the particles of a group of particles are spherical and also assuming that the particles in a given group of particles all have substantially the same size (available surface area), the rate of dissolution of a group of particles can be readily determined. Using this information a formulation can be created with different groups or types of particles wherein each group of particles has a known drug release profile within the environment the formulation is delivered to. The formulation preferably comprises a number of different groups which release drug at different rates and/or times and provide a desired drug release profile, e.g. substantially constant levels in the surrounding area over a therapeutically effective time period.

Calculations are provided below in Tables 3, 4 and 5 respectively for total volumes of 1 cm³, 0.5 cm³ and 0.1 cm³ which are volume sizes that might be used for typical dosages of orally administered pharmaceutically active compounds.

TABLE 3
Total volume is 1 cm³

| | | | | Surface |
|---|---|---|---|---|
| | | | Surface | area |
| number of | radius | Diameter | area | Volume |
| spheres | (micrometers) | (micrometers) | (cm²) | (cm⁻¹) |
| 1 | 6203.5 | 12407.0 | 4.84 | 4.8 |
| 10 | 2879.4 | 5758.8 | 10.42 | 10.4 |
| 100 | 1336.5 | 2673.0 | 22.45 | 22.4 |
| 1,000 | 620.4 | 1240.7 | 48.36 | 48.4 |
| 10,000 | 287.9 | 575.9 | 104.19 | 104.2 |
| 100,000 | 133.7 | 267.3 | 224.47 | 224.5 |
| 1,000,000 | 62.0 | 124.1 | 483.60 | 483.6 |
| 10,000,000 | 28.8 | 57.6 | 1041.88 | 1041.9 |
| 100,000,000 | 13.4 | 26.7 | 2244.66 | 2244.7 |
| 1,000,000,000 | 6.2 | 12.4 | 4835.98 | 4836.0 |
| 10,000,000,000 | 2.9 | 5.8 | 10418.79 | 10418.8 |
| 100,000,000,000 | 1.3 | 2.7 | 22446.61 | 22446.6 |

TABLE 4
Total volume is 0.5 cm³

| | | | Surface | Surface |
|---|---|---|---|---|
| number of | Radius | diameter | area | area |
| spheres | (micrometers) | (micrometers) | (cm2) | Volume |
| 1 | 4923.7 | 9847.5 | 3.05 | 6.1 |
| 10 | 2285.4 | 4570.8 | 6.56 | 13.1 |
| 100 | 1060.8 | 2121.6 | 14.14 | 28.3 |
| 1,000 | 492.4 | 984.7 | 30.46 | 60.9 |
| 10,000 | 228.5 | 457.1 | 65.63 | 131.3 |
| 100,000 | 106.1 | 212.2 | 141.40 | 282.8 |
| 1,000,000 | 49.2 | 98.5 | 304.65 | 609.3 |
| 10,000,000 | 22.9 | 45.7 | 656.34 | 1312.7 |
| 100,000,000 | 10.6 | 21.2 | 1414.05 | 2828.1 |
| 1,000,000,000 | 4.9 | 9.8 | 3046.47 | 6092.9 |
| 10,000,000,000 | 2.3 | 4.6 | 6563.43 | 13126.9 |
| 100,000,000,000 | 1.1 | 2.1 | 14140.48 | 28281.0 |

TABLE 5
Total volume is 0.1 cm³

| | | | Surface | Surface |
|---|---|---|---|---|
| number of | radius | Diameter | area | area |
| spheres | (micrometers) | (micrometers) | (cm2) | Volume |
| 1 | 2879.4 | 5758.8 | 1.04 | 10.4 |
| 10 | 1336.5 | 2673.0 | 2.24 | 22.4 |
| 100 | 620.4 | 1240.7 | 4.84 | 48.4 |
| 1,000 | 287.9 | 575.9 | 10.42 | 104.2 |
| 10,000 | 133.7 | 267.3 | 22.45 | 224.5 |
| 100,000 | 62.0 | 124.1 | 48.36 | 483.6 |
| 1,000,000 | 28.8 | 57.6 | 104.19 | 1041.9 |
| 10,000,000 | 13.4 | 26.7 | 224.47 | 2244.7 |
| 100,000,000 | 6.2 | 12.4 | 483.60 | 4836.0 |
| 1,000,000,000 | 2.9 | 5.8 | 1041.88 | 10418.8 |
| 10,000,000,000 | 1.3 | 2.7 | 2244.66 | 22446.6 |
| 100,000,000,000 | 0.6 | 1.2 | 4835.98 | 48359.8 |

### Adhesives

The tape of the invention may be coated with an adhesive and that adhesive may coat all or a portion of one on both sides of the tape. By placing adhesive on one side of the tape the adhesive can serve to adhere the tape to the screw or other implant it is used with. The adhesive may be on both sides adhering the tape to screw or other implant on one side and adhering the screw or other plant to a bone and/or other implanted device on the other side. When both sides of the tape are coated the adhesive on one side may be the same or different from the adhesive on the other side. It may, for example, be desirable for one adhesive to more or less biodegradable as compared to the adhesive on the other side. A wide range of adhesives are known and many are commercially available. Adhesives used with the present invention to coat on a tape and/or adhere particles in holes on implants may be pressure sensitive. Further, they should be biocompatible and may be biodegradable.

Pressure sensitive adhesive (PSA) compositions are well known in the art and many are commercially available. Generally, silicone PSA's are produced by either blending or condensing a silicone resin and a polydiorganosiloxane. These types of silicone pressure sensitive adhesives have been disclosed for example in U.S. Pat. Nos. 2,736,721, 2,814,601, 2,857,356, and 3,528,940. The adhesion of such materials can be varied by altering the ratio of units in the resinous material or the ratio of resin to polydiorganosiloxane.

The use of silicone based PSAs in devices where they have been shown not to cause an adverse reaction with humans is also known in the art. Examples of such devices can be found in U.S. Pat. Nos. 3,731,683 (Zaffaroni); U.S. Pat. No. 3,797,494 (Zaffaroni); U.S. Pat. No. 4,031,894 (Uhrquhart, et al.) and U.S. Pat. No. 4,336,243 (Sanvordeker, et al.). Such devices are held in contact with the skin by means of a pressure-sensitive adhesive and the devices are typically intended to be left in contact with the skin for a period of 24 hours or more. In view of the extended period of contact with the skin which is sensitive to irritation, the adhesives should be capable of adhering to a metal screw or other implant device of the invention without causing an immune or inflammatory response when implanted in or on bone.

Silicone pressure-sensitive adhesives are known to be non-iritating and non-sensitizing to the skin and have been used as adhesives for certain devices such as those for the controlled release of nitroglycerine. Silicone PSAs are known in the art and are commercially available.

U.S. Patent No. RE 35,474 teaches a transdermal drug delivery device for the controlled delivery of amino-functional drugs. This patent teaches that aminofunctional drugs interfere with the properties of PSAs by catalyzing the reaction of silicon bonded hydroxyl (silanol) groups and, thereby, cause increased increase shear of the PSA material and, thus, loss of tack during storage. The patent teaches that this effect can be inhibited by chemically treating the PSAs with an agent which reduces their silanol content. The '474 patent, however, teaches that the amount of agent used to treat the PSA must be an amount of at least 0.8 moles triorganosilyl units for every mole of silanol. U.S. Pat. No. 6,337,086 indicated that this amount of treating agent generally reduces the silanol concentration of the PSA to levels below about 7700. The '086 indicates that when the silanol content is too low, further cross-linking of the adhesive network is sufficiently reduced and this loosely cross-linked network becomes plasticized and oozy, thus resulting in poor PSA quality.

Accordingly, the '086 patent teaches silicone PSAs having a low, but well defined concentrations of silanol with a wider range of users in terms of drugs that may be added for transdermal delivery.

### Particle Formation Methodlogy

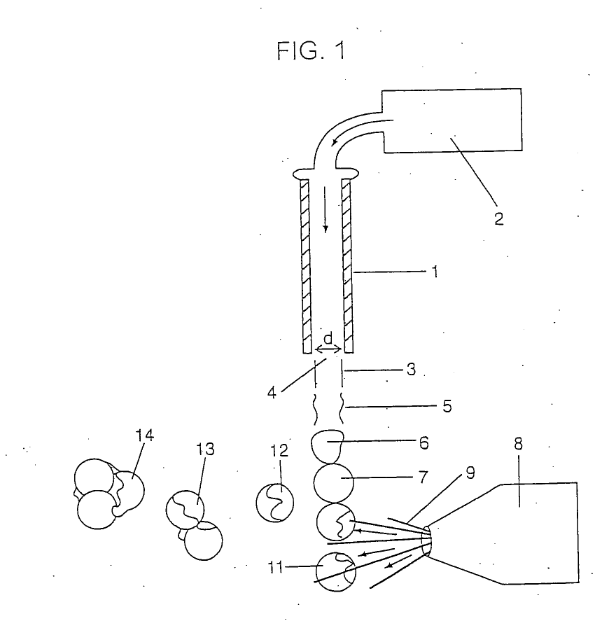

Particles and coated particles can be produced via any available technology. Referring to FIG. 1, cylindrical tube 1 is shown in fluid connection with a liquid source 2 which can supply liquid 3 to the tube 1. The liquid 3 exits the tube 1 from an exit opening which can be any configuration but is preferably circular and has a diameter D. The liquid 3 exits the opening 4 and forms a stream which breaks into segments 5 and eventually forms partial spheres 6 and then spheres 7 which are substantially equal in size and shape. The spheres 7 could be used in creating a group of particles for attachment to a device such as a surgical screw. Different size spheres from different sized tubes 1 could create different groups of spheres as needed for a desired dissolution profile.

The processing of FIG. 1 can stop at the formation of the particles 7. However, in order to attempt to obtain a dissolution profile which achieves a longer steady state level of the desired compound a coating is often used. The coating source 8 creates a spray 9 of a coating material which is brought into contact with and sticks to particles 10, 11 and 12 often in different amounts. Further, two particles 13 may become coated together or three or more particles 14 may become coated together.

The result is a random and unknown mixture of particles coated to different degrees and combined with different numbers of other particles. Although coated particles of this type could be used to attach to a device such as a screw they are not preferred because of the random and unknown nature of the resulting mixture of coated particles. The coating material can be mixed with rather than sprayed on the particles and a similar random mixture of coated particles and coated groups of particles will result. The random mixture has some advantages. It can provide a greater range of release rates than a single type of particle. The greater range of release rates may provide a release profile which is desirable. However, considerable trial and error is required in producing a desired release profile. Further, because the size distribution is unknown great care must be taken once the desired profile is obtained in repeating all preparation steps precisely from batch to batch. Otherwise, each new batch of formulation produced will have a different release profile.

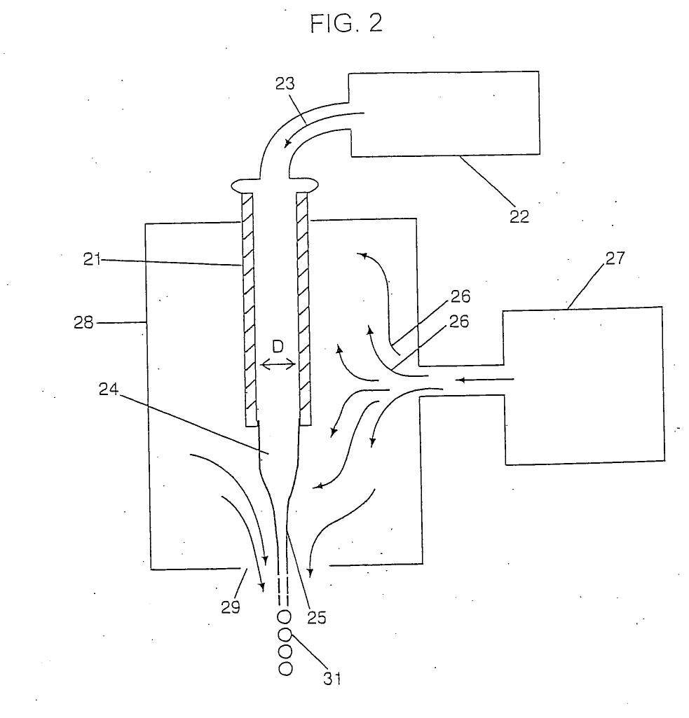

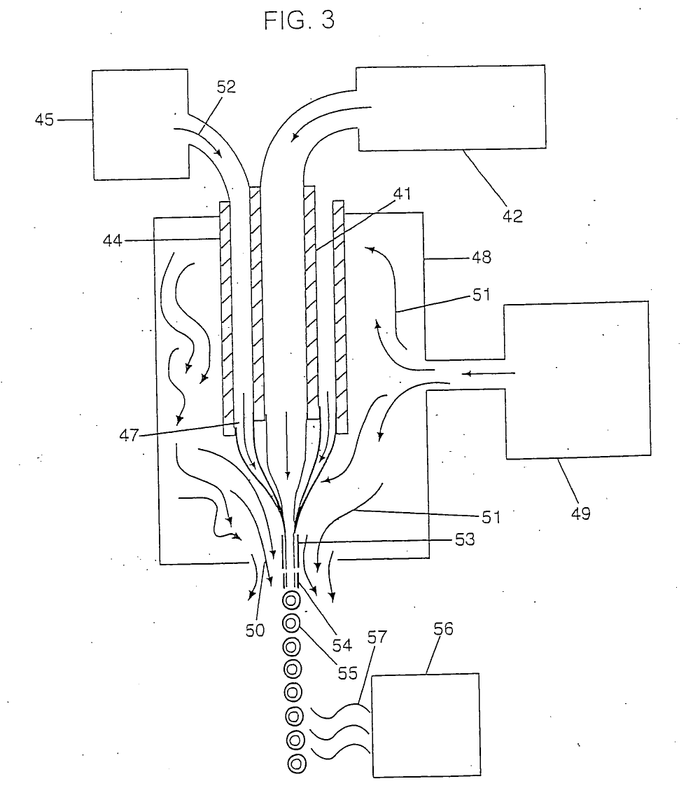

The process for producing particles 7 as shown in FIG. 1 has yet another disadvantage or limitation. Specifically, the diameter D of the tube 1 dictates that the diameter of the particles 7 formed will be approximately D×1.89 (Rayleigh, “On the instability of jets”, Proc. London Math. Soc., 4-13, 1878). Thus, when attempting to make very small particles (e.g. less than 20 micrometers) the inside diameter of the tube 1 must be very small. Not only is it difficult to manufacture tubes with such a small diameter but the narrower tubes tend to clog easily. These problems can be solved by using a different technology for producing particles and coated particles as shown in FIGS. 2 and 3.

FIG. 2 shows a tube 21 supplied by a liquid source 22. The liquid 23 flows out of the exit 24. The liquid 23 stream is focused to a narrowed stable jet 25 by a gas 26 provided by the gas source 27 flowing into a pressure chamber 28 and out of an exit orifice 29. The jet 25 disassociates into segments 30 which form spheres 31 in the same manner in which the stream of liquid 3 forms the spheres 7 shown in FIG. 1. However, the spheres 31 have a diameter which is 1.89× the diameter Dⱼ of the jet and not 1.89× the diameter D of the tube 21. The diameter of the jet 25 (Dⱼ) is substantially smaller than the diameter D of the tube 21. Thus, the system of FIG. 2 can be used to make very small particles as compared to the system of FIG. 1 without clogging the exit 24 of the tube 21 because the diameter D of the tube 21 can remain large—and without clogging the exit orifice 29 of the pressure chamber 28 because the jet 25 exits the orifice 29 surrounded by the gas 26.

The particles 31 can be coated using a spray on coating as shown in FIG. 1. However, similar problems occur as described above with reference to FIG. 1. The particles 31 can be used without any coating. Groups of particles can be combined to provide a desired dissolution profile. The small size of the particles provides certain advantages as shown in Tables 1-5. Particles in a size range of 1-20 micrometers can not be easily produced in a system as shown in FIG. 1 and particles in this size range provide the greatest differences in surface areas—see Tables 1-5 and Table 2 in particular. However, the particles themselves (without a coating) are limited in terms of the dissolution profile they can produce particularly when the total volume of the particles in a formulation is limited. Thus, a coating is preferred and a preferred means of obtaining such is shown in FIG. 3.

The system schematically shown in FIG. 3 includes a tube 41 in fluid connection with a liquid source 42 which supplies liquid 43 to the cylindrical channel of the tube 41. A tube 44 is concentrically positioned around the tube 41 and is in fluid connection with a coating source 45. The exit opening 46 of the tube 41 and the exit opening 47 of the tube 44 are both positioned inside of a pressure chamber 48. The chamber 48 is in fluid connection with the gas source 49 which flows out of the exit orifice 50 of the chamber 48. The gas 51 focuses the streams of liquid 43 and coating 52 into a stable jet 53. The jet 53 disassociates into segmented streams 54 of liquid 43 concentrically surrounded by coating 52. The segmented streams 53 form spheres 55. The spheres 55 are comprised of a liquid 43 center surrounded by a polymeric (e.g. PLGA) coating 52. The spheres 55 are preferably very small, e.g. a diameter of less than 50 μm, preferably less than 20 μm and more preferably about 10 μm. The smaller the particles the more readily evaporation will take place which will cure or solidify the coating 52.

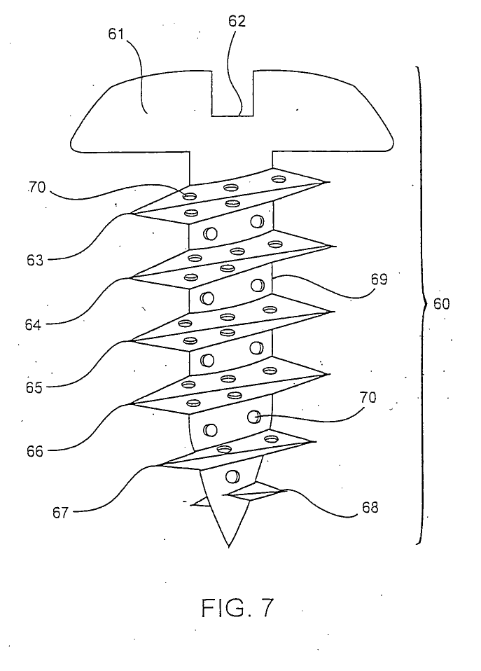

An energy source 56 may be used to direct energy 57 onto the particles 55 to enhance the rate of curing, hardening, evaporation, etc. The energy 57 may be any type of energy including heat, forced air, I.R. or U.V. light etc. alone or in combination. Some polymer materials are designed to be cured using a particular frequency of light. The light can be directed, focused and/or intensified using lenses, mirrors and the like to obtain a desired result. The particles 55 could be produced and a biocompatible adhesive used to bind the particles to indentations as shown on the screws in FIGS. 7-10. Alternatively, the spheres could be produced and directed into the indentation on the device and cured in place.

The coated particles 55 can include any liquid 43 coated with any coating material 52. However, in accordance with the present invention it is preferable that the liquid 43 be comprised of a pharmaceutically active drug which is preferably an antimicrobial and more preferably an antibiotic. Further, the coating material can be comprised of any type of material which can be cured, dried or fixed in any fashion in order to form an outer spherical coating around the center. However, it is preferable that the coating material be comprised of a polymer material and more preferable if the polymer material is quickly and readily curable and is a material which is commonly accepted as useful as a carried material in controlled release formulations used in pharmaceutical applications.

Both the particles and the tape are comprised of a biocompatible, biodegradable material. There are a number of known materials that can be used and many are both commercially available and approved by governmental agencies around the world for use in humans.

Examples of syntehtic biocompatible, biodegradable polymers which can be used in making the tape and or the coated particles of the invention are listed below:
poly(d1-lactide)
poly(1-lactide)
polyglycolide
poly(dioxanone)
poly(glycolide-co-trimethylene carbonate)
poly(1-lactide-co-glycolide)
poly(d1-lactide-co-glycolide)
poly(1-lactide-co-d1-glycolide)
poly(glycolide-co-trimethylene carbonate-co-dioxanone)
poly(vinyl alcohol)
poly(anhydride)
poly(orthoester)
poly(-caprolactone)
poly(dioxanone).-caprolactone).

Natural or natural processed polymers such as processed collagen can be used to the tape.

It should be noted that the particles and the tape need not be comprised of a single polymer and that the tape made be comprised of a different polymer or polymer than the particles are comprised of. Further, when multiple tapes are used together they may be of different thicknesses and/or be comprised of different polymers. This may be done to create particles and tapes which biodegrade in situ at different rates. Thus, controlled release rates of the drug such and the antibiotic and/or anticoagulant can be obtained.

In addition to the polymeric materials mentioned above other materials can be used. For example, a number of such polymer materials are disclosed within the patents and publications described below.

U.S. Pat. No. 3,773,919 describes creating slow release formulations producing a steady release of drug in the bloodstream by employing polylactide-drug mixtures in the dosage form. The inventors describe using a chemical based microencapsulation procedure for forming precipitates of the polylactide-drug mixtures suitable for injection. They discuss many potential applications for their invention including the administration of morphine.

U.S. Pat. No. 4,942,035 describes using PLGA polymer as an excipient allowing formulations to be created to facilitate the controlled release of polypeptide active drugs into solutions.

U.S. Pat. No. 5,514,380 describes modifying the cross-linking in PLGA polymer in order to obtain more controllable release profiles.

U.S. Pat. No. 5,543,158 describes potential benefits of using PLGA polymer with pharmaceutically active drug to create particles in a very small size range to minimize incorporation of the injected formulation into the patient's macrophages which would result in inactivation of the drug.

U.S. Pat. No. 5,650,173 describes an emulsion system for creating particles of PGLA and active drug suitable for injection.

U.S. Pat. No. 5,654,008 describes a technique for combining PLGA and active drug into microparticles suitable for injection by using an emulsion system created using a static mixer.

U.S. Pat. No. 5,759,583 describes using a quaternary ammonium surfactant as an excipient to facilitate the creation of PLGA drug combinations suitable for injection to create a controlled release formulation.

U.S. Pat. No. 5,912,015 describes using metal cations as release modulators in the injectable drug formulation comprising PLGA and active drug.

U.S. Pat. No. 5,916,598 describes using emulsion systems and solvent extraction techniques as tools for creating microparticles comprised of PLGA and active drug for sustained release formulations.

U.S. Pat. No. 6,254,890 describes using PLGA to create sustained release formulations containing nucleic acids.

Previous approaches for combining PLGA with active drug to create such controlled release formulations relied on chemical techniques for creating microparticles suitable for injection. These techniques have focused on the use of solvent systems to produce emulsions resulting in the creation of a precipitate of crystalline microparticle in an approximate size range suitable for injection. Other systems involve removing solvents used during the fabrication process. The US FDA as well as international drug regulatory authorities have drafted regulations strictly limiting the amount of residual solvent acceptable in marketed pharmaceutical preparations (ICH Harmonized Tripartite Guideline Q3C Impurities: “Guidelines for Residual Solvents”).

Additional discussion of categories of systems for controlled release may be found in Agis F. Kydonieus, Controlled Release Technologies: Methods, Theory and Applications, 1980 (CRC Press, Inc.).

Controlled release drug delivery systems may also be categorized under their basic technology areas, including, but not limited to, rate-preprogrammed drug delivery systems, activation-modulated drug delivery systems, feedback-regulated drug delivery systems, and site-targeting drug delivery systems.

In rate-preprogrammed drug delivery systems, release of drug molecules from the delivery systems is “preprogrammed” at specific rate profiles. This may be accomplished by system design, which controls the molecular diffusion of drug molecules in and/or across the barrier medium within or surrounding the delivery system. Fick's laws of diffusion are often followed.

In activation-modulated drug delivery systems, release of drug molecules from the delivery systems is activated by some physical, chemical or biochemical processes and/or facilitated by the energy supplied externally. The rate of drug release is then controlled by regulating the process applied, or energy input.

In feedback-regulated drug delivery systems, release of drug molecules from the delivery systems may be activated by a triggering event, such as a biochemical substance, in the body. The rate of drug release is then controlled by the concentration of triggering agent detected by a sensor in the feedback regulated mechanism.

In a site-targeting controlled-release drug delivery system, the drug delivery system targets the active molecule to a specific site or target tissue or cell. This may be accomplished, for example, by a conjugate including a site specific targeting moiety that leads the drug delivery system to the vicinity of a target tissue (or cell), a solubilizer that enables the drug delivery system to be transported to and preferentially taken up by a target tissue, and a drug moiety that is covalently bonded to the polymer backbone through a spacer and contains a cleavable group that can be cleaved only by a specific enzyme at the target tissue.

Another controlled release dosage form is a complex between an ion exchange resin and the lipoates. Ion exchange resin-drug complexes have been used to formulate sustained-release products of acidic and basic drugs. In one preferable embodiment, a polymeric film coating is provided to the ion exchange resin-drug complex particles, making drug release from these particles diffusion controlled. See Y. Raghunathan et al., Sustained-released drug delivery system I: Coded ion-exchange resin systems for phenylpropanolamine and other drugs, J. Pharm. Sciences 70: 379-384 (1981).

Injectable micro spheres are another controlled release dosage form. Injectable micro spheres may be prepared by non-aqueous phase separation techniques, and spray-drying techniques. Micro spheres may be prepared using polylactic acid or copoly(lactic/glycolic acid). Shigeyuki Takada, Utilization of an Amorphous Form of a Water-Soluble GPIIb/IIIa Antagonist for Controlled Release From Biodegradable Micro spheres, Pharm. Res. 14:1146-1150 (1997), and ethyl cellulose, Yoshiyuki Koida, Studies on Dissolution Mechanism of Drugs from Ethyl Cellulose Microcapsules, Chem. Pharm. Bull. 35:1538-1545 (1987).

To form a coated particle 55 the liquid 43 is forced through the channel of the tube 41. The liquid is preferably a relatively high concentration of a drug such as an antibiotic in either an aqueous or alcohol based solvent or other solvent which will quickly evaporate (e.g. ether). The exit opening 46 of the tube 41 and the exit opening 47 of the tube 44 are both positioned inside the pressure chamber 48. The coating material 52 is initially in a liquid form and is forced through the exit opening 46 of the tube 44 which is positioned concentrically around the tube 41 in a manner which causes a stream of the liquid coating material to be expelled from the opening 47 at substantially the same velocity as the liquid 43 is forced from the opening 46 of the tube 41. Accordingly, the stream of the coating material is concentrically positioned around the stream of the center liquid 43. The streams exit the openings of the two concentrically positioned tubes as a single combined stream which then disassociates into segments streams 53 which segments form the cooled spheres 55.

In order for the spheres to be made small it is necessary to use the gas from the gas source 49 forced into the pressure chamber 48 in a manner which causes the gas to exit the pressure chamber 48 downstream of the concentrically positioned streams exiting the tubes 41 and 44. It is preferable for the density of the liquid 43 to be substantially the same as the liquid of the coating 52. This allows the gas from the gas source 49 to focus the concentrically positioned streams into a stable unified jet which flows out of the chamber 48 breaking up into segments and thereafter forming the spherical coated particles 55 of the coating material surrounding the center of pharmaceutically active drug.

In accordance with the invention the gas from the gas source forms the stable jet and the diameter of the jet is substantially smaller than would be the case if the gas were not focusing the streams exiting the tubes 41 and 44. The diameter of the jet is defined by the following formula:

$${d}_{j} ≅ {( \frac{8 {ρ}_{1}}{{π}^{2} Δ {P}_{g}} )}^{1 / 4} {Q}^{1 / 2}$$

wherein dⱼ is the diameter of the stable unified jet, □ indicates approximately equally to where an acceptable margin of error is ∀ 10%, ρ₁ is the average density of the liquid of the jet and ΔP_{g} is change in gas pressure of gas surrounding the stream at a given point A at the exit and Q is the total flow rate of the stable unified jet.

By using the technology described above and shown in FIGS. 2 and 3 it is possible to form very small and very uniform particles. The particles may be of any size but are preferably in less than 100 micrometers in diameter, more preferably less than 50 micrometers in diameter and still more preferably less than 20 micrometers in diameter. The technology described above and shown in FIGS. 2 and 3 is capable of producing particles which are as small as approximately 1 micrometer in diameter and preferred devices of the invention will include particles which have a diameter of approximately 10 micrometers. The sphere forming technology can produce particles which are substantially identical in shape (spherical) and substantially identical in size ±10% variation in the particle diameter, more preferably ±3% and still more preferably ±1% variation in particle diameter where the particle may have a diameter as small as 1 μm or more or as large as 100 μm or more.

Those skilled in the art will understand that in addition to the tubes 41 and 44 a plurality of additional concentrically positioned tubes may be added to the system. This would make it possible to add additional coating materials or include additional active components surrounded by outer shells of coating material. An out coating of adhesive could be added so that the particles 55 have an adhesive thereon and adhere to the indentations in the screws as shown in FIGS. 7-10. Those skilled in the art will understand that the system works best when the Weber Number is in a range of from about 1 to about 40 wherein the Weber Number is defined by the following equation:

$$We = \frac{{ρ}_{g} {V}_{g}^{2} d}{γ}$$

wherein the ρ_{ε} is the density of the gas, d is the diameter of the stable microjet, γ is the liquid-gas surface tension and V_{g}² is the velocity of the gas squared. More preferably the Weber number is in a range of about 5 to about 25.

Further, those skilled in the art will understand that it is preferable for the Ohnesorge number to be less than 1, wherein the Ohnesorge number (Oh) is defined by

$$Oh = \frac{{μ}_{l}}{{( {ρ}_{l} γ d )}^{1 / 2}}$$

wherein μ₁ is the velocity of the liquid, ρ₁ is the density of the liquid and d is the diameter of the stable capillary microjet.

Those skilled in the art will also understand that the method for producing particles and coated particles as described above is best carried out when the difference in the pressure between the pressure chamber exit orifice is equal to or less than 20 times the surface tension of the liquid comprising the coating material with the gas, divided by the radius of the stable unified jet. Details relating to the technology are described within issued U.S. Pat. No. 6,234,402 issued May 22, 2001 and incorporated herein by reference. Those skilled in the art will understand that some adjustments may be made in the density and velocity of the different fluids and gases used in order to obtain the desired result in terms of the fluid—fluid interfaces including the particle interface between the coating material and the inner liquid material as well as the stable interface between the gas and the coating material. It is desirable to obtain the stable microjet stream which has substantially no aberrations or pertubations in the stream making it possible for the stream to disassociate into very uniform size and shaped particles. This systems shown in FIGS. 2 and 3 make it possible to maintain a stable liquid-gas interface between the outer surface of the liquid or coating material and the gas thereby forming a stable jet which is focused on the exit orifice of the pressure chamber resulting in particles which have very small deviation in terms of diameter from one particle to the next. It is also possible to create hollow particles and to reverse the positioning of the different fluids. For examples, the center tube can be used to supply gas whereas the pressure chamber can be used to supply a liquid. The technology for such is described within issued U.S. Pat. No. 6,196,525 issued Mar. 6, 2001 which patent along with other patents cited herein is incorporated in its entirety.

### Dissolution Profiles

When any particle dissolves in any solvent the amount of solute in the solution increases over time. However, some solvents are present in systems where the portion of the dissolving solute is being removed from the solution. This could take place in a chemical reaction where a portion of the dissolved solute reacts with another components present in the system. However, the most typical situation is where a drug present in an area and diffuses away from that area which subtracts solute drug from the surrounding area. In any such system the dissolution profile over time shows an increase followed by a steady state followed by a decrease as is shown by the solid line in FIG. 4. It is desirable to maintain the level of a drug above the therapeutic level shown by the line of short dashes but below a toxic level shown by the line of long dashes or level where addition drug provides no additional benefit. Maintaining the level of drug in a desired range for a significant period is difficult to obtain particularly when using a single type of particle.

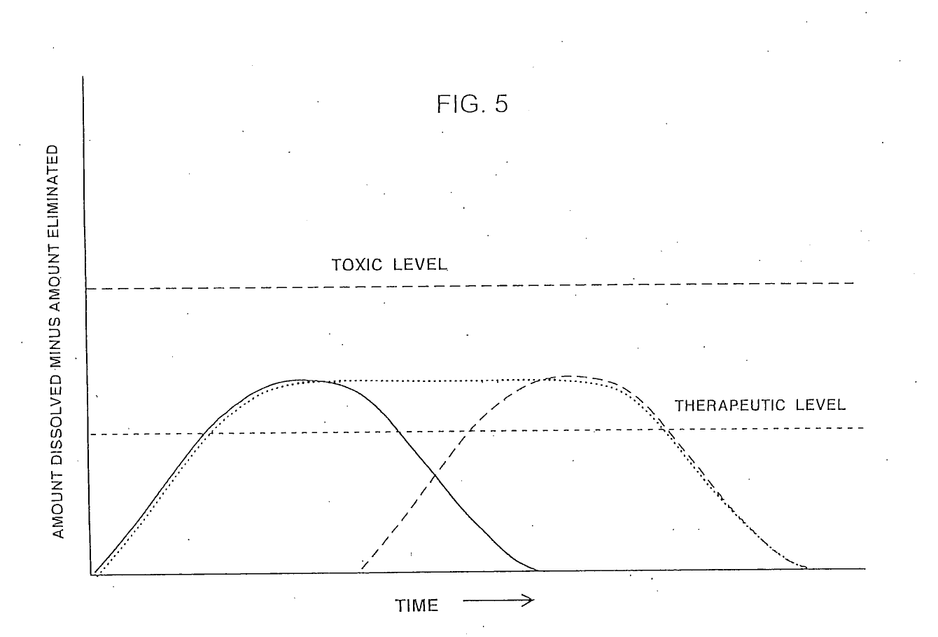

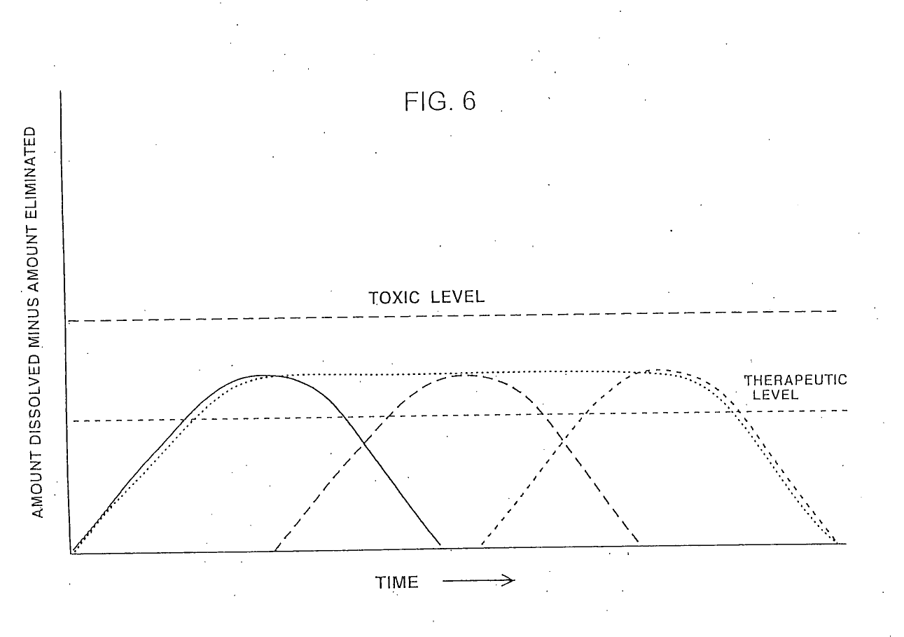

FIG. 5 shows how the therapeutic level can be maintained over a longer period of time using two different types of particles. In FIG. 5 the independent effect of a first type of particle is shown by the solid line. The dashed curve shows the independent effect of a second type of coated particle. The dotted curve shows the combined effect of the two types of particles. When the particle of the first type are completely dissolved and are being metabolized out of the system the coatings on the particle of the second type have dissolved and the rate of dissolution matches the rate at which all drug in the system is being diffused out of the desired area. Thus, a longer steady state period is maintained. This effect is further enhanced using three different types of particles as shown in FIG. 6.

Controlled release within the scope of this invention can be taken to mean any one of a number of extended release dosage forms. The following terms may be considered to be substantially equivalent to controlled release, for the purposes of the present invention: continuous release, controlled release, delayed release, depot, gradual release, long-term release, programmed release, prolonged release, proportionate release, protracted release, repository, retard, slow release, spaced release, sustained release, time coat, timed release, delayed action, extended action, layered-time action, long acting, prolonged action, repeated action, slowing acting, sustained action, sustained-action medications, and extended release. Further discussions of these terms may be found in Lesczek Krowczynski, Extended-Release Dosage Forms, 1987 (CRC Press, Inc.).

There are corporations with specific expertise in drug delivery technologies including controlled release oral formulations such as Alza corporation and Elan. A search of patents, published patent applications and related publications will provide those skilled in the art reading this disclosure with significant possible controlled release technologies. Examples include the technologies disclosed in any of the U.S. Pat. No. 5,637,320 issued Jun. 10, 1997; U.S. Pat. No. 5,505,962 issued Apr. 9, 1996; U.S. Pat. No. 5,641,745 issued Jun. 24, 1997; and U.S. Pat. No. 5,641,515 issued Jun. 24, 1997. Although specific technologies are disclosed here and in these patents the invention is more general than any specific technology. This includes the discovery that by placing pharmaceutically active drug in a controlled release particle groups which maintain therapeutic levels over substantially longer periods of time as compared to quick release formulations, improved unexpected results are obtained.

Particles Formed Using Supercritical Fluid Precipitation

The devices, systems and methodology disclosed and described above in connection with FIGS. 2 and 3 can also be used in combination with supercritical fluid precipitation technology of the type described within U.S. Pat. No. 6,063,910 issued May 16, 2000; U.S. Pat. No. 5,766,637 issued Jun. 16, 1998; U.S. Pat. No. 6,228,394 issued May 8, 2001; and U.S. Pat. No. 6,095,134 issued Aug. 1, 2000 all of which are incorporated herein by reference in their entirety. Basically, the technology utilizes a supercritical fluid such as liquid CO₂ in order to form solid particles of a material such as a drug or a protein for use in a formulation.

Referring to FIG. 2 the gas source 27 could be replaced with a liquid CO₂ and the liquid CO₂ could become the focusing fluid. The liquid 23 supplied into the tube 21 could be any liquid comprised of any desired material. However, the liquid 23 would preferably be a liquid which included an active compound such as a drug which is dissolved within a solvent such as water and further combined with a solvent such as ethanol. The solvent liquid 23 is focused by the surrounding liquid 26 which may be CO₂. When the CO₂ exits the pressure chamber 28 via the orifice opening 29 the rapid evaporation draws the liquid water and ethanol away leaving dry particles 31.

Referring to FIG. 3 it would also be possible to use supercritical fluids in place of the coating 52 or in place of the gas 51. Those skilled in the art will recognize that a variety of different combinations of liquids, gases, solutions and supercritical fluids are possible using the systems as shown and described above with respect to FIGS. 2 and 3 particularly when taken in combination with the above-referenced patents which disclose basic technology used in the field of supercritical fluid precipitation.

### Surgical Implant

A typical surgical implant or screw 60 is shown in FIG. 7. The screw 60 includes a top 61 which includes an indentation 62 which can be used for placing the screw 60 into a bone (not shown). The screw includes ridges 63, 64, 65, 66, 67 and 68 and a shaft 69. Both the shaft 69 and ridges 63-68 include circular indentations 70. These indentations may be created in any manner such as by the use of a conventional drill or by the use of a laser. Alternatively, the indentations may be formed within the screw when it is created. The indentations are generally circular in shape and are generally of a size in a range of from about 1 micron to about 50 microns in diameter or 5 to 20 microns and may all be substantially the same size or vary in size to match groups of particles.

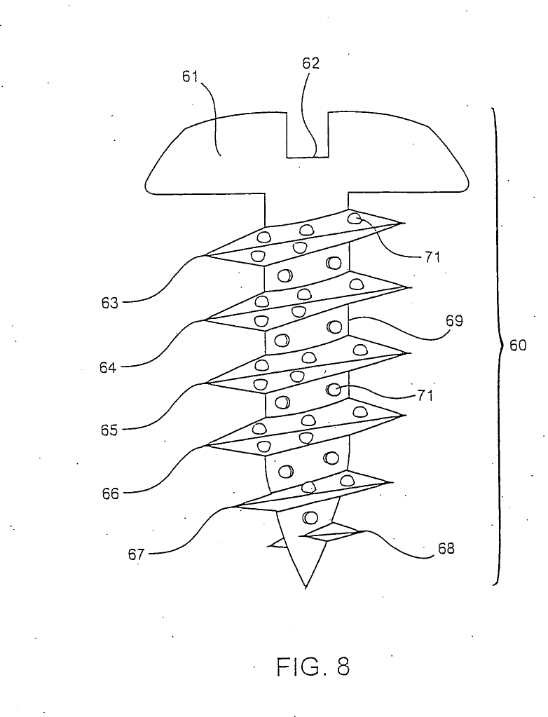

The screw 60 is also shown in FIG. 8. However, in FIG. 8 each of the indentations 70 has a spherical particle 71 positioned therein. The particles 71 are shown protruding outward here for visualization. However, the particles 71 are preferably positioned such that they do not extend beyond the outer surface of the screw. Accordingly, when the screw is screwed into a bone the particles 71 are not broken apart.

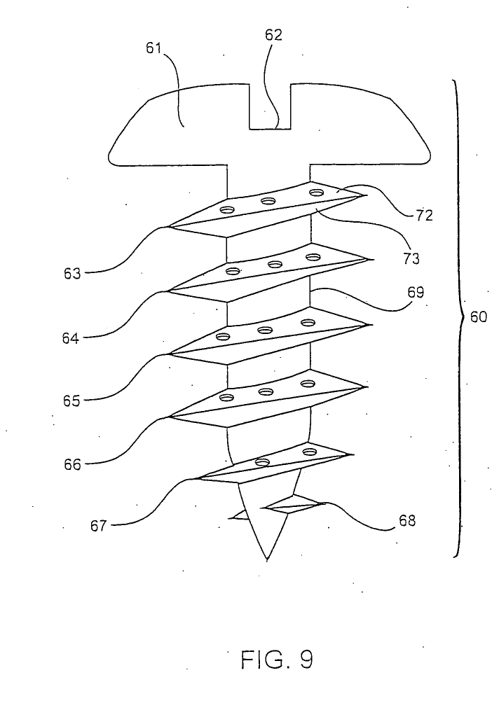

FIG. 9 shows the screw 60 with the indentations 70 only in the ridges 63-68. More specifically, the indentations 70 are only on the upper surface 72 of each ridge and not on the lower surface 73. This is done in that the lower surface 73 is subjected to greater stress when the screw 60 is screwed into the bone.

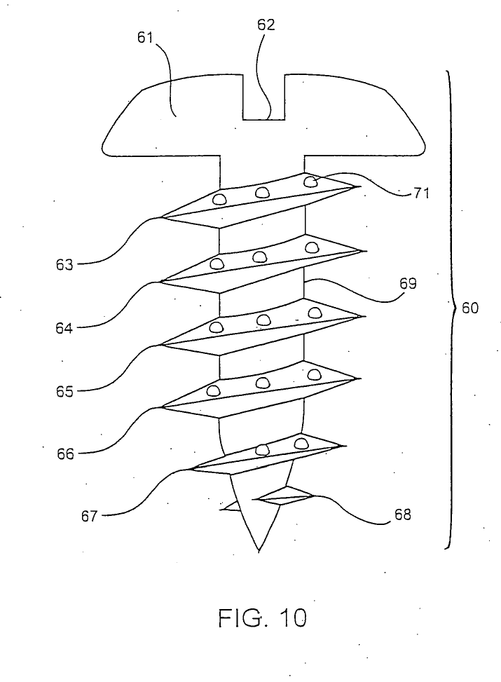

FIG. 10 shows the screw of FIG. 9 with particles 71 positioned in the indentations. With this configuration it is possible for the particles 71 to protrude outward slightly in that they are not subjected to substantial stress. However, it is preferable for the particles to be positioned such that they do not extend beyond the surface into which they are inserted.

The FIGS. 7-10 all refer to and show surgical screws. However, other types of surgical implants and bandages can be used in connection with the present invention. Screws that are used come in a variety of different lengths. For example, the screw could come in a length of from 5 mm to 50 mm and a shaft diameter in a range of approximately 2 mm to 20 mm. A typical surgical screw could have a length of 12.5 mm and a shaft diameter of about 3 mm. The surface area of such a screw would be (π)(3)(12.5)=120 mm². If holes were drilled with a diameter of approximately 80 mircrometers and a depth of approximately 4 times that or 320 micrometers. The volume of each hole can be calculated as

$$( \frac{π}{4} ) {( 80 \times {10}^{- 3} )}^{2} ( 4 \times 80 \times {10}^{- 3} ) = 1.6 \times {10}^{- 3} {mm}^{2} .$$

The area if each hole is approximately

$$\frac{π}{4} {( 80 \times {10}^{- 3} )}^{2} = 5 \times {10}^{- 3} {mm}^{2} .$$

If approximately 10% of the screw area is covered with holes the number of holes can be calculated as (N)(5×10⁻³ mm²)=(0.1)(120)mm² or N=˜2000 holes.

The volume of 2000 holes is calculated by (2000)(1.6×10⁻³)=3.2 mm³. If a sphere has a drug volume/space volume ratio of 0.7 and a packing density of approximately 80% the volume of the stored drug can be calculated as (3.2 mm³)(0.7)(0.8)=1.8 mm³ of drug.
1.8 mm³=1.8 mg of drug per screw.

As indicated above the size of the screw could be varied. Further, the percentage area of the screw having holes therein could vary from approximately 5% to 50% or more of the surface area. Further, the diameter and the depth of the holes could also be varied greatly to obtain larger or smaller amounts of the drug as needed. It is important to note that the amount of drug provided here is the amount of drug which is provided to the immediate area surrounding the screw. When drug is administered systemically only a very small amount of drug would actually reach the immediate environment surrounding the screw. Thus, even small amounts of antimicrobial agents such as 1.8 mg would generally be far more than would reach the surrounding area if larger doses such as 1000 mg were administered systemically. Accordingly, an advantage of the present invention is that it provides for site specific delivery of the antimicrobial agent.

### Types of Drugs and Implants

The invention is not limited to screws but can be applied to all types of devices using all types of antimicrobial, antibacterial, antifungal, and antiviral compounds including those compounds and devices described in the following US patents:

U.S. Pat. No. 6,582,715—Antimicrobial orthopedic implants; U.S. Pat. No. 6,579,539—Dual mode antimicrobial compositions; U.S. Pat. No. 6,565,913—Non-irritating antimicrobial coatings and process for preparing same; U.S. Pat. No. 6,365,220—Process for production of actively sterile surfaces; U.S. Pat. No. 6,361,731—Method of forming a temporary implant; U.S. Pat. No. 6,361,567—Non-irritating antimicrobial coating for medical implants and a process for preparing same; U.S. Pat. No. 6,361,526—Antimicrobial tympanostomy tube; U.S. Pat. No. 6,267,782—Medical article with adhered antimicrobial metal; U.S. Pat. No. 6,238,686—Anti-microbial coating for medical devices; U.S. Pat. No. 6,190,407—Medical article with adhered antimicrobial metal; U.S. Pat. No. 6,155,812—Cement mold for a temporary implant; U.S. Pat. No. 6,113,636—Medical article with adhered antimicrobial metal; U.S. Pat. No. 6,080,490—Actively sterile surfaces; U.S. Pat. No. 6,017,553—Anti-microbial materials; U.S. Pat. No. 6,013,106—Medical article with adhered antimicrobial metal ions and related methods; U.S. Pat. No. 5,985,308—Process for producing anti-microbial effect with complex silver ions; U.S. Pat. No. 5,984,905—Non-irritating antimicrobial coating for medical implants and a process for preparing same; U.S. Pat. No. 5,980,974—Coated orthopaedic implant components; U.S. Pat. No. 5,958,440—Anti-microbial materials; U.S. Pat. No. 5,945,153—Non-irritating antimicrobial coating for medical implants and a process for preparing same; U.S. Pat. No. 5,855,950—Method for growing an alumina surface on orthopaedic implant components; U.S. Pat. No. 5,837,275—Anti-microbial materials; U.S. Pat. No. 5,770,255—Anti-microbial coating for medical devices; U.S. Pat. No. 5,753,251—Anti-microbial coating for medical device; U.S. Pat. No. 5,695,857 Actively sterile surfaces; U.S. Pat. No. 5,681,575—Anti-microbial coating for medical devices; U.S. Pat. No. 5,674,293—Coated orthopaedic implant components; U.S. Pat. No. 5,593,438—Intraocular lens with metallic coatings for preventing secondary cataracts; U.S. Pat. No. 5,534,288—Infection-resistant surgical devices and methods of making them; U.S. Pat. No. 5,522,840—Device for the non-surgical seal of the interstice in the wall of a vessel; U.S. Pat. No. 5,454,886—Process of activating anti-microbial materials; U.S. Pat. No. 5,152,993—Method of preparing an implant body for implantation; U.S. Pat. No. 5,123,927—Method and apparatus for antibiotic knee prothesis; U.S. Pat. No. 4,615,705—Antimicrobial surgical implants.

### Heterogenous Particle Formulations

Devices of the present invention (such as the screw 60 show in FIGS. 7-10) have bound to them a plurality (2 or more) of groups of different types of particles. A first group of spherical particles is present wherein each particle of the first group has a same diameter as other particles in the group with a margin of error in terms of particle diameter size of approximately ±10% or less. The formulation then includes a second group of spherical particles wherein each particle of the second group has the same diameter as the other particles in the second group with a margin of error of about ±10% or less. The particles within the first group are different from the particles within the second group and preferably have a difference in terms of the steady state levels which difference is sufficient to provide a longer steady state level of antimicrobial to the surrounding area than either of the groups by themselves. Preferably, the first group of particles and the second group of particles each comprise 100 or more particles, more preferably a 1,000 of more particles, and still more preferably 10,000 or more particles and may comprise 10⁵ to 10¹⁰ or more particles.

Although the heterogeneous groups of particles bound to a device can be produced using particle formation technology of various types the technology as described above with respect to FIGS. 2 and 3 are preferred in that they produce very uniform sized and shaped particles. Further, the particles may be solid spheres which may be produced using the technology as shown in FIG. 2. However, the preferred device of the invention includes a group of particles wherein the particles are coated using the technology as shown within FIG. 3. Preferably, the device such as a screw 60 is bound to 3 or more groups of spherical particles wherein the particles within each group are the same and are different between the groups. Further, preferred devices will be bound to at least some particles which are not coated e.g. a first group of particles with no coating and a relatively small particle size. Thus, the first group of particles will provide for substantially immediate dissolution and release of all of the compound or drug which is present in the particles. This causes the drug to quickly reach a therapeutic level in the desired surrounding area. The remaining groups of particles are coated and remain undissolved. When a known amount of time has passed diffusion will have removed from the surrounding area (e.g. the bone) a sufficient amount of the drug added by the first group such that the concentration of the drug in the surrounding area is beginning to decline, the coating on the second group of particles will then dissolve so that the second group of particles now begins to add drug to the surrounding area thereby gradually increasing the concentration via the second group of particles at a rate substantially corresponding to the rate at which drug from the first group of particles is being diffused out. This is shown within the graph of FIG. 5. The process can be repeated several times with several different groups of particles and three different groups of particles are shown within the graph of FIG. 6 and may be bound to the screw 60 as shown in FIGS. 7-10.

In a particularly preferred embodiment of the invention an antimicrobial is dissolved in a solvent which may be water, ethanol or a combination of water and ethanol. The solution of drug in the solvent is then coated with a polymer material which can be quickly cured by the addition of energy or evaporation as shown within FIG. 3. Thus, a group of particles is formed wherein the particles are comprised of a liquid center which liquid is comprised of a solution of drug and solvent in an outer core of polymer material which is substantially inert i.e. does not provide a pharmacological effect. Such particles are produced in a variety of different size ranges. Each size is used to produce a group of particles which, by itself, is sufficient to provide for therapeutic levels of a drug to the area surrounding the implant e.g. the screw 60 of FIGS. 7-10. When the coating dissolves the liquid within the spheres, which is a liquid drug (e.g. a drug in an aqueous solution) is immediately released. When the drug has diffused away to the point of beginning to drop below therapeutic levels the next group of particles with a thicker coating have dissolved to the point where the drug within these particles is released raising the level of drug in the surrounding area. By including a plurality of different groups it is possible to maintain the therapeutic level of the drug over a long period of time e.g. 1 day, several days (2 to 6 days) to 1 week, and even several weeks (2 to 3 weeks) to 1 month.

Those skilled in the art will recognize that variability in terms of the rate at which the coating material dissolves can be changed by increasing the thickness of the coating and/or by changing the composition of the coating material as some materials will dissolve more quickly than others. Accordingly, the different groups of particles within the formulation may be particles which are all of the same size, but have different coating thicknesses. Alternatively, the particles may be all of the same size, and have the same coating thicknesses but have different coating compositions from one group to another wherein the composition of coating on one group of particles dissolves more rapidly than the coating composition on another group within the formulation.

### Tape Dispenser

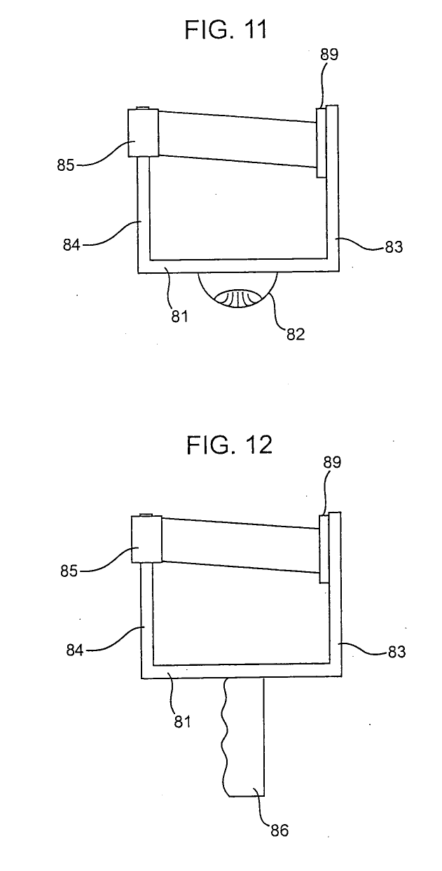

An aspect of the invention is a tape dispenser 80 as shown in FIG. 11. The dispenser 80 is comprised of “c” shaped frame 81 having a finger holdable tab 82 attached at a surface. The frame 81 has a break-away anchor tab 89 on one arm 83 and a free rotating cylinder of tape 85 on the end of the arm 84.

As shown in FIG. 12 the finger tab 82 can be a hand grip 86 for use in wrapping larger screws or implants.

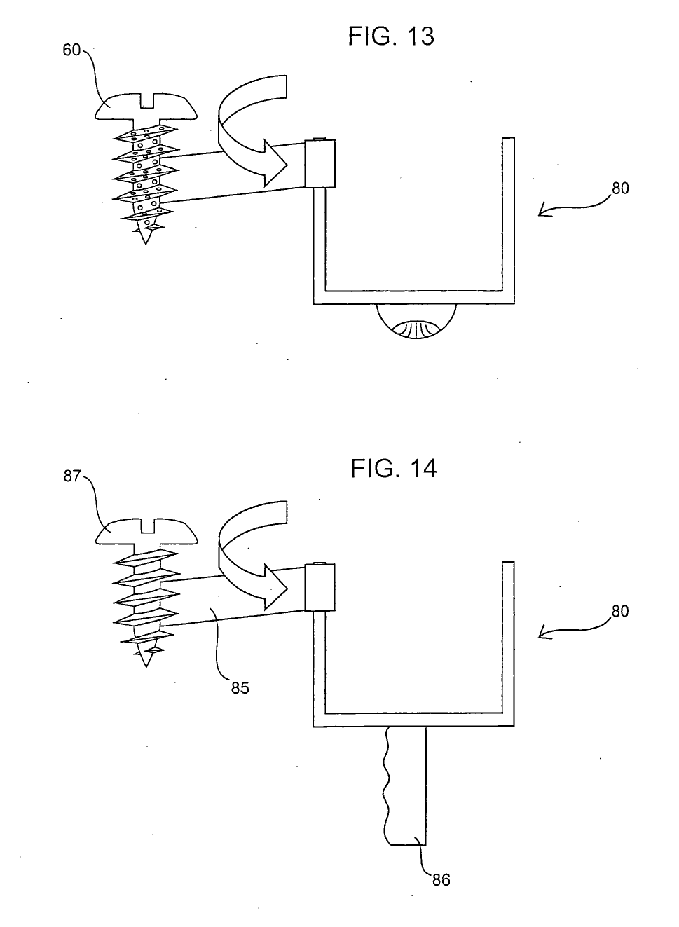

As shown in FIG. 13 the dispenser 80 is being used to wrap a screw 60 as shown in FIG. 8.

As shown in FIG. 14 the hand-held dispenser as shown in FIG. 12 can be used to wrap tape 85 on a conventional screw 87.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Those skilled in the art will recognize that the technology described here can be provided to a number of different types of drugs and to heterogenous formulations of all different numbers of particle groups. However, here a specific example is described wherein the active drug is first included within particles which have no coating and thereafter are included within two additional groups of particles wherein the percent thickness of the spheres is varied.

| Capsule | Sphere Diameter | | |
|---|---|---|---|
| Thickness | 5 microns | 10 microns | 20 microns |
| 0 | S/V = 2.4 | S/V = 1.2 | S/V = 0.6 |
| 10% | S/V = 4.7 | S/V = 2.3 | S/V = 1.2 |
| 30% | S/V = 38 | S/V = 19 | S/V = 9.4 |

The surface area to volume ratio numbers in Table 6 must be taken in the context of the capsule thickness. Microspheres with a capsule thickness of zero are composed entirely of active drug; there is by definition no inactive ingredient forming a capsule layer. Therefore, even though a 10 μm microsphere with zero capsule thickness has the same surface area to volume ratio (1.2) as a 20 μm microsphere with a 10% capsule thickness, release of active drug from the 20 μm sphere will occur only after the outer layer has dissolved whereas active drug from the 10 μm sphere in this example will begin to be released as soon as microsphere dissolution begins.

In addition, in the context of this invention, high surface area to volume values do not necessarily mean faster release of active drug into the area surrounding the implant. This is because, for the case of non-zero capsule thickness microspheres, the outer material is an inactive ingredient.

By having a formulation in which a distinct capsule thickness is present in microspheres of a distinct size, a true programmable controlled release profile can be engineered by selecting (a) the capsule thickness and microsphere size and (b) by selecting in which proportions different populations of microspheres selected in (a) are combined and bound to the implant (e.g. screw) or other device.

For example, a slow release antibiotic formulation bound to indentations on a screw could consist of ⅓ zero capsule thickness 5 μm microspheres for rapid release, ⅓ 10% capsule thickness 10 μm spheres for intermediate release and ⅓ 10% capsule thickness 20 μm microspheres for long term release as part of a single formulation. Because the capsule of inactive material must be largely dissolved before active drug release, this approach has the distinct advantage of minimizing the overlap of delivery by the various formulation components. This allows the aggregate PK profile of the formulation to be formed by superposition of the release profiles of the components of the formulation.

### Example 2

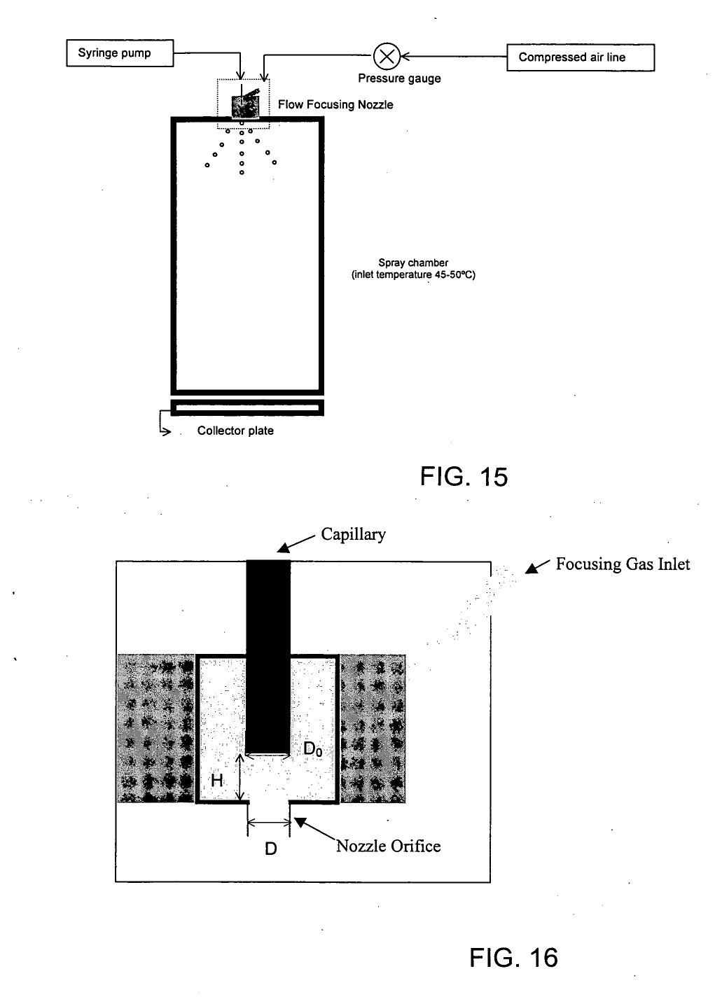

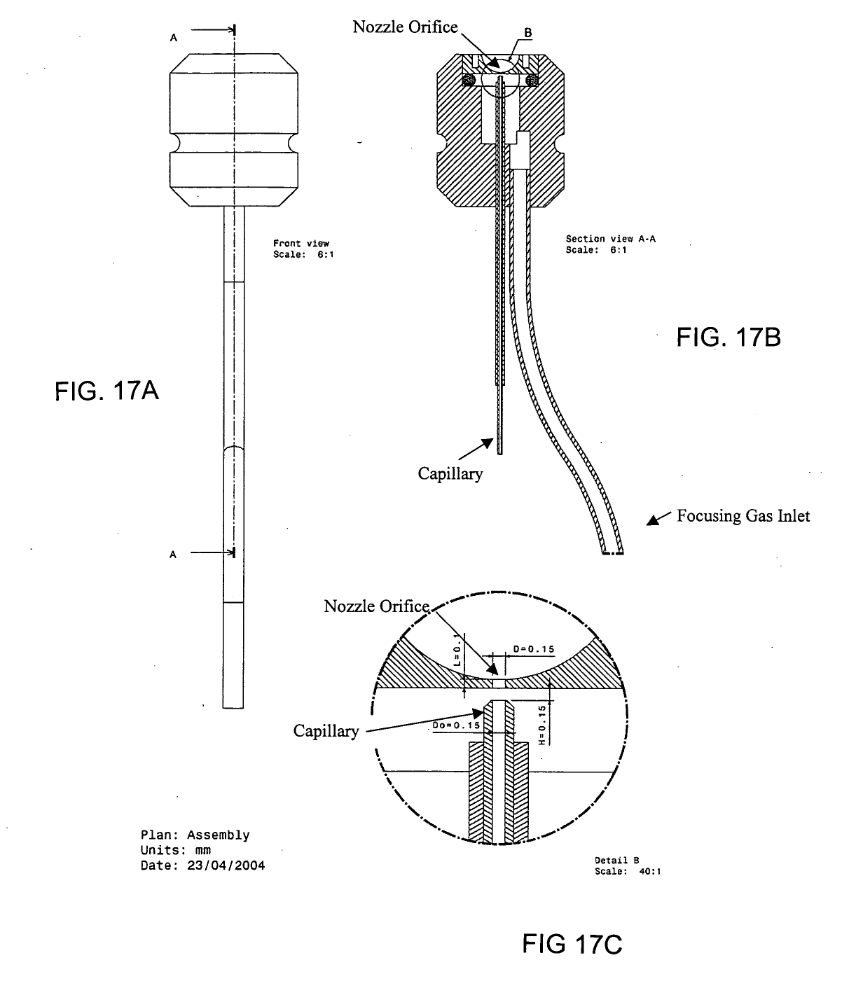

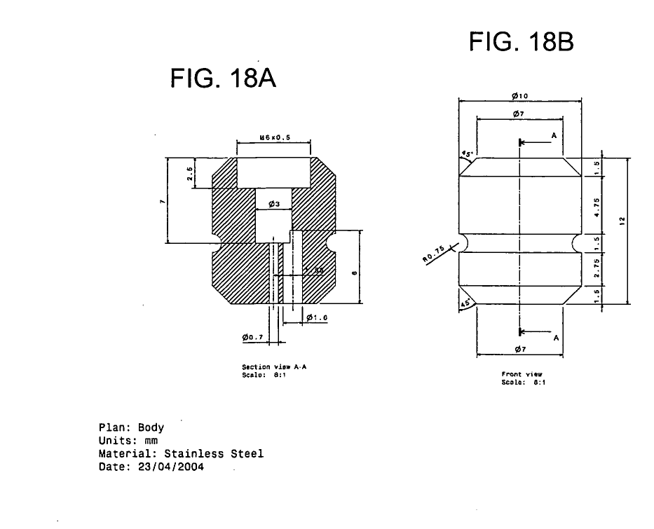

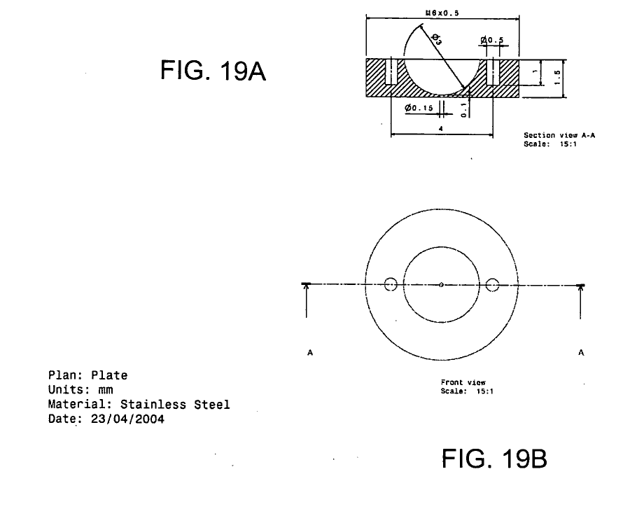

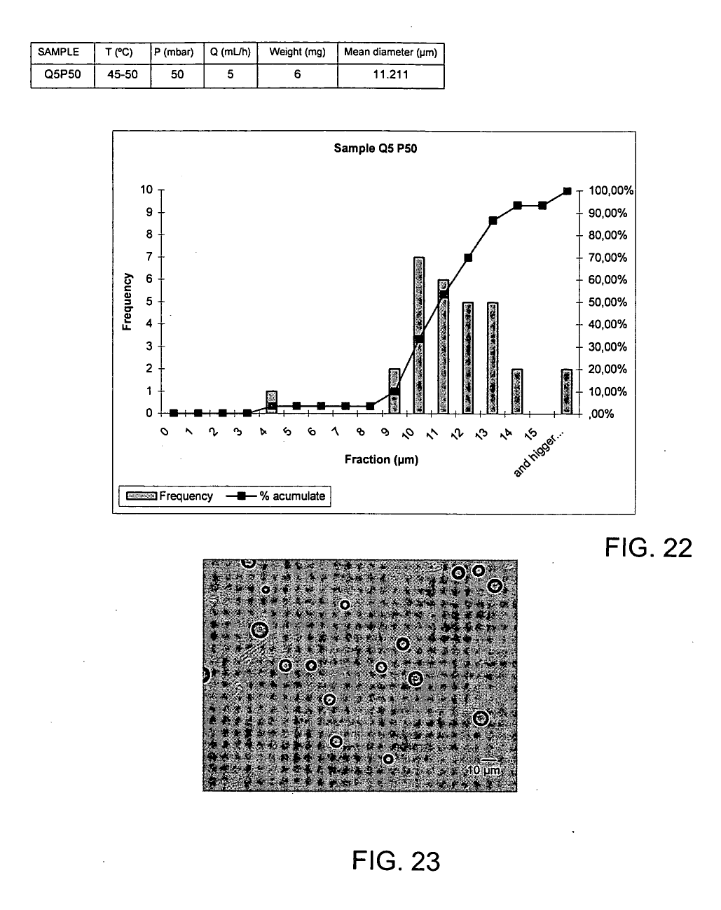

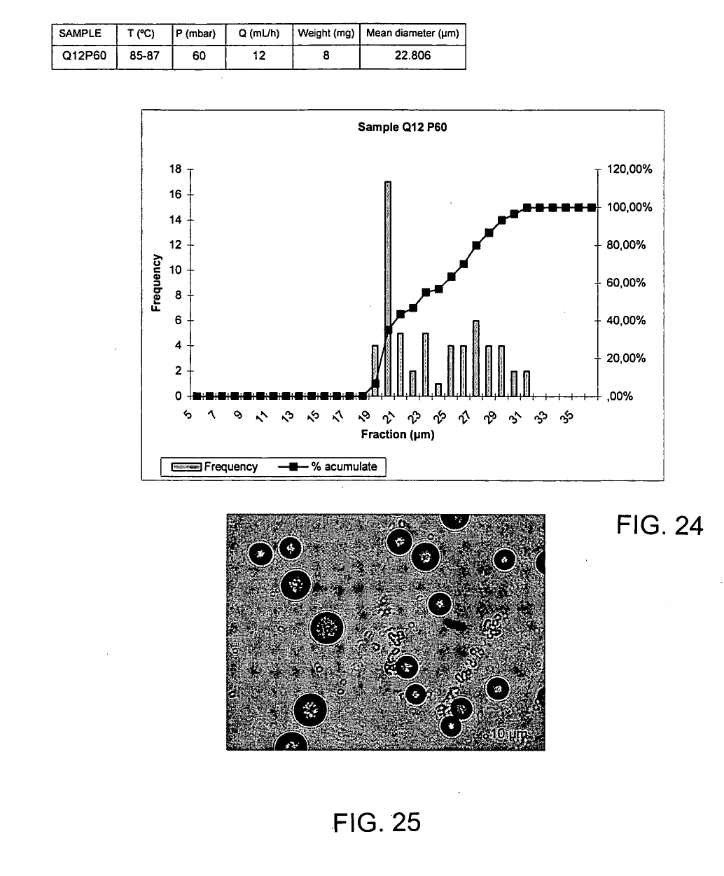

A specific configuration of the Flow Focusing nozzle system was used to fabricate a series of microencapsulated GS/PLGA micro spheres (FIGS. 15-29). The device consists of a compartment with a gas inlet supply (providing the focusing gas), a nozzle orifice opposite a capillary tube connected to a syringe pump which conveys a continuous flow of water-in-oil emulsion to a fixed point in front of the nozzle orifice.

The stream of gas across the nozzle orifice creates a funnel shaped “lens-of-air” into which the liquid from the capillary is introduced. This results in a steady jet of water-in-oil emulsion exiting the orifice. This jet breaks up quickly into equal-sized drops whose size is a function of the pressure drop across the orifice and the flow rate through the capillary tube. Finally, these liquid drops, made of a water-in-oil emulsion, are dried in a hot chamber to obtain the desired microencapsulated GS/PLGA micro spheres.

Different amounts of GS (Aldrich) were dissolved in phosphate buffer (pH 6.2). The pH was adjusted to 6.5-7 with NaOH 4N to facilitate GS solubility. This solution was dispersed in a polymer solution in ethyl acetate (Panreac) (4% w/v of PLGA (Resomer® RG 502 (Boehringer Ingelheim) by ultrasonication (Ultrasons, P Selecta) at room temperature for 15 min.

To preserve the emulsion stability for over time, 1.1% w/v of the surfactant Span® 60 was added to the oil phase.

The resulting water-in-oil emulsion was sprayed inside a chamber with inlet temperature 45-50° C. for samples Q2P170 and Q2P50 and 85-87° C. for sample Q12P60. To obtain the smallest micro particles (sample Q2P170) the distance between capillary tube and exit orifice (H, see FIG. 16) was modified to 125 μm.

The obtained particles were collected at the bottom of the chamber as a dry powder on a plate (FIG. 15).

TABLE 1
GS/PLGA micro sphere sample production data using the Flow Focusing nozzle.

| | | | | Encapsulation | |
|---|---|---|---|---|---|
| | Production | | Entrapment² (%) | Efficiency³ (%) | |
| Sample Label | conditions¹ | Mean diameter (μm) | mean ± SD | mean ± SD | Production⁴ (%) |
| Q2P170 | Q = 2 ml/hr | 6.691 | 10.952 | 32.860 | 10.17 |
| | P = 170 mbar | | | | |
| Q5P50 | Q = 5 ml/hr | 11.211 | 20.637 ± 0.160 | 61.917 ± 0.480 | 27.01 |
| | P = 50 mbar | | | | |
| Q12P60 | Q = 12 ml/hr | 22.806 | 14.233 ± 0.064 | 42.704 ± 0.192 | 25.42 |
| | P = 60 mbar | | | | |
| ¹Production Conditions refer to Q, the flow rate through the capillary feeding the water-oil emulsion of GS/PLGA into the Flow Focusing nozzle as determined by the syringe pump setting and P, the pressure drop across the Flow | | | | | |
| # Focusing nozzle plate as determined by the plate orifice size and the focusing gas flow rate. | | | | | |

²Entrapment is defined as (mg drug × 100)/(mg micro spheres).
³Encapsulation Efficiency is defined as (mg micro sphere drug × 100)/(mg initial drug).
⁴Production Percent is defined as (mg micro spheres) × 100/(mg GS + mg PLGA + mg surfactant).

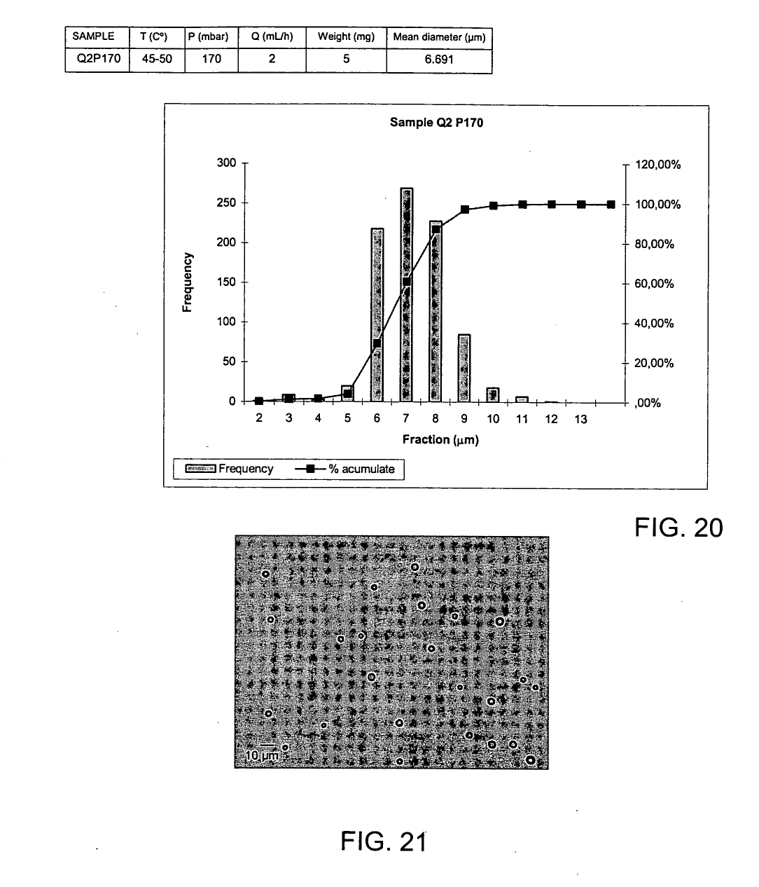

The FIGS. 20-25 illustrate the particle size distributions for the three sizes of GS/PLGA micro spheres as fabricated.

### Example 3

Fabricated GS/PLGA micro spheres were studied in-vitro for their GS release characteristics. GS/PLGA micro spheres were first incubated in phosphate buffered saline (PBS), with changes of buffer at fixed time points. The antimicrobial activity of the supernatants was determined by placing the supernatants into small open cylinders (Oxford cups) that rest on the surface of an agar plate previously seeded with a bacterial culture (Serratia marcescens, ATCC No. 29632). The plate is then incubated for 36 hours. If Gentamicin sulfate is present in sufficient concentration, the bacteria cannot grow and a clear zone of inhibition (ZOI) is the result.

One milligram of each size of GS/PLGA micro spheres was placed in a 1.5 mL eppendorf tube to which was added 125 microliters of PBS. All tubes were incubated at room temperature with gentle agitation.

At 2, 4, 6, and 8 hours, all tubes were given a 10 second centrifugation, after which 100 microliters was removed from each tube, retained for analysis, and the volume replaced with 100 microliters of fresh PBS. After the final time point, the tubes were again centrifuged for 30 seconds and all residual liquid (taking care not to remove any GS/PLGA micro spheres) was removed and discarded. The GS/PLGA micro spheres were re-suspended in 100 microliters of PBS and immediately added to an Oxford cup, ensuring complete re-suspension and delivery of the GS/PLGA micro spheres to the Oxford cup.

Overnight cultures of each strain of bacteria were diluted in PBS to approximate a very dilute suspension equal to a 0.5 MacFarland Standard. A swab of each suspension was prepared and a Mueller-Hinton agar plate, 140 mm in diameter, was inoculated using the swab in a back and forth motion to completely cover the surface of the plate.

Oxford cups (the cut end of a 200 microliter pipette tip) were placed on freshly seeded plates, into which was then placed 100 microliters of each time point. The plates were allowed to incubate at room temperature without disturbance for 36 hours.

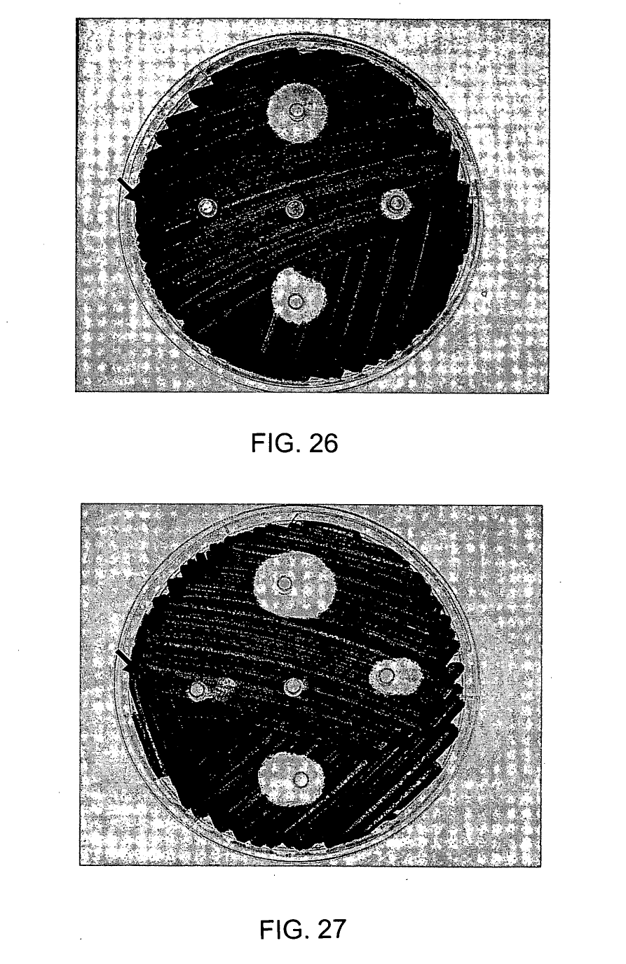

Zones of inhibition (ZOI) were photographed (see FIGS. 26, 27 and 28) with a Canon Eos 300D digital camera to make a permanent recording of the experiment. S. marcescens was photographed using a background of white light to demonstrate its characteristic pigment production.

For all photographs, at the top of each plate is indicated the type/size of GS/PLGA micro sphere used. The ZOI created at the 2 hour time point is at the top, the 4 hour time point at the bottom, the 6 hour time point to the right, and the 8 hour time point to the left. The residual GS/PLGA micro spheres is in the middle of the plate. The arrow in all the photos below indicates the ZOI created by the 8 hour time point specimen.

Agar plate photographs showing ZOIs are shown in FIGS. 26, 27 and 28.

FIG. 26—The ZOI are shown as clear areas. The number ‘5’ at the top indicates the use of Q2P170 (7 μm sphere size) GS/PLGA micro spheres. The numbers 2, 4, 6, and 8 indicate the time points in hours. The lower case ‘f’ in the center indicates the residual GS/PLGA micro spheres collected after the last time point was taken.

FIG. 27—The ZOI are shown as clear areas. The number ‘6’ at the top indicates the use of Q5P50 (11 μm sphere size) GS/PLGA micro spheres. The numbers 2, 4, 6, and 8 indicate the time points in hours. The lower case ‘f’ in the center indicates the residual GS/PLGA micro spheres collected after the last time point was taken.

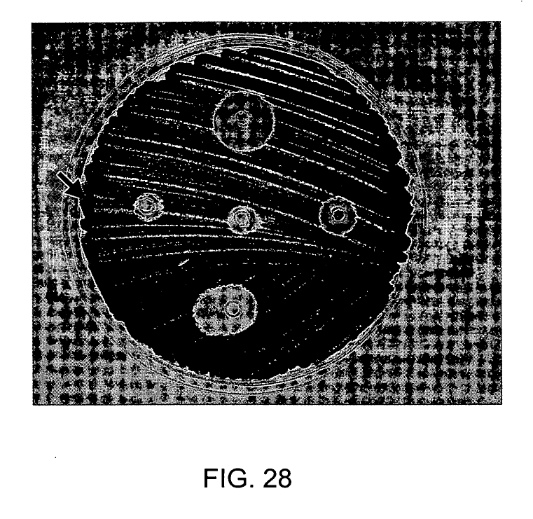

FIG. 28—The ZOI are shown as clear areas. The number ‘8’ at the top indicates the use of Q 12P60 (23 μm sphere size) GS/PLGA micro spheres. The numbers 2, 4, 6, and 8 indicate the time points in hours. The lower case ‘f’ in the center indicates the residual GS/PLGA micro spheres collected after the last time point was taken.

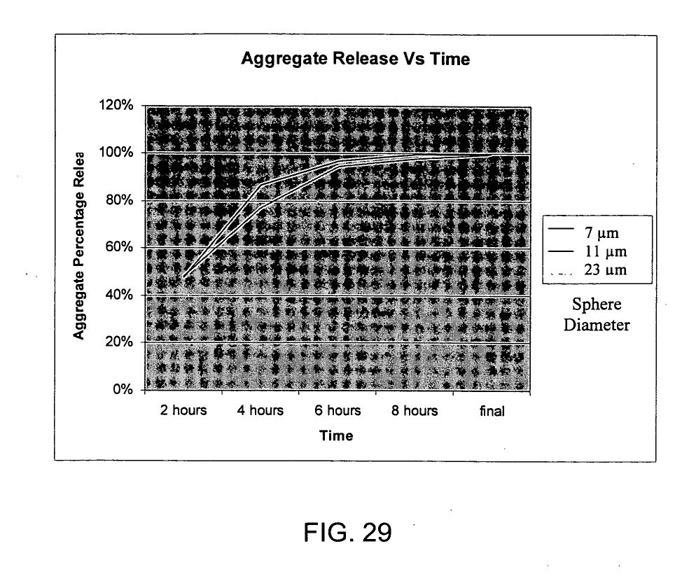

The relative amount of inhibition was determined by cutting out and weighing a picture of each ZOI. The results of this analysis of the relative amounts of inhibition versus time for size of micro sphere studied is shown in the Table below. FIG. 29 shows this data of the below Table expressed in graphic form.

| | Sphere | 2 | 4 | 6 | 8 | | |
|---|---|---|---|---|---|---|---|
| Specimen | Size | hours | hours | hours | hours | Final | Total |
| Q2P170 | 7 μm | 11.9 | 9.7 | 2.4 | 0.8 | 0.0 | 24.8 |
| ZOI | | 48.0% | 39.1% | 9.7% | 3.2% | 0.0% | 100.0% |
| % of total | | | | | | | |
| liberated | | | | | | | |
| Q5P50 | 11 μm | 19.8 | 11.8 | 7.0 | 1.4 | 0.8 | 40.8 |
| ZOI | | 48.5% | 28.9% | 17.2% | 3.4% | 2.0% | 100.0% |
| % of total | | | | | | | |
| liberated | | | | | | | |
| Q12P60 | 23 μm | 14.2 | 11.9 | 5.2 | 2.9 | 3.2 | 37.4 |
| ZOI | | 38.0% | 31.8% | 13.9% | 7.8% | 8.5% | 100.0% |
| % of total | | | | | | | |
| liberated | | | | | | | |

The above Table shows the relative Rate of Release of GS from GS/PLGA micro spheres and PCFD. Data points determined by integrating ZOI areas.

FIG. 29—ZOI integrals plotted as a function of time expressed as aggregate amount released. The smaller GS/PLGA micro spheres are seen to release GS faster than the larger GS/PLGA micro spheres.

FIG. 29 clearly illustrates the fact that the smaller GS/PLGA micro spheres release GS more completely at earlier time points than larger GS/PLGS micro spheres. Using this information, specific combinations of different sizes of GS/PLGA micro spheres could be combined to produce a specific, desired GS release profile.

The release profile illustrated occurs over a several hour period. This is in the context of placing the GS/PLGA micro spheres in a large (relative) volume of water. If the spheres are applied into the static environment of a surgical incision site and, in particular, in contact with implantable orthopedic hardware, the release profile can be expected to be more prolonged.

The release profile can be expected to further lengthen if the GS/PLGA micro spheres were combined with bio degradable media such as a collagen or polymer tape which could be applied to orthopedic hardware before implantation. As an alternative to tape, the GS/PLGA micro spheres could be mixed into liquid fibrin sealant such as Baxter FloSeal, CoSeal or Tisseal prior to placement into the surgical site.

Another vehicle into which GS/PLGA micro spheres could be incorporated prior to introduction into the surgical site includes an emulsion allowing the GS/PLGA micro spheres to be introduced into the surgical site as a foam.

In the cases of a biodegradable tape, fibrin sealant or emulsion, a selection of GS/PLGA micro spheres in specific sizes can be pre-determined to facilitate a desired release profile of GS into the surgical site. Desired release profiles include release profiles that provide rapid release of some GS within hours, intermediate release of GS within days and long term release of GS for weeks following implantation. Such release profiles could be expected to reduce the incidence of surgical site infection in patients having introduction of implantable orthopedic hardware for fractures and disorders of the spine in addition to other applications.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A tape, comprising:
a biodegradable strip of material;
an antibiotic; and
an adhesive.

2. The tape of claim 1, wherein the step of material is polymeric material and the antibiotic is embedded in the polymeric material.

3. The tape of claim 1, wherein the antibiotic is adhered to the adhesive.

4. A method, comprising the steps of:
wrapping a surgical implant with a biodegradable tape comprising an antibiotic; and
placing the wrapped implant in a patient.

5. The method of claim 4, wherein the implant is chosen from a bar, a plate and a surgical screw and the tape covers at least a portion of the implant.

6. The method of claim 4, wherein the tape further comprises an adhesive coated on a first side of the tape.

7. A layered tape, comprising:
a first strip of polymeric material;
a second strip of polymeric material; and
an antibiotic positioned between the first and second strips.

8. The tape of claim 1, wherein the antibiotic comprises:
a first group of spherical particles wherein each particle of the first group has the same diameter as other particles in the first group with a margin of error of ±10% or less;
a second group of spherical particles wherein each particle of the second group has the same diameter as other particles in the second group with a margin of error of ±10% or less.

9. The tape of claim 8, wherein the first group and the second group each comprise 100 or more particles and further wherein particles of the first group dissolve at a rate which is faster than a rate at which the particles of the second group dissolve.

10. The tape of claim 8, wherein the antibiotic further comprises:
a third group of spherical particles wherein each particle of the third group has the same diameter as other particles in the third group with a margin of error of ±20% or less.

11. The tape of claim 10, wherein the third group comprises 100 or more particles and further wherein particles of the third group dissolve at a rate different from a rate at which the particles of the first and second groups dissolve.

12. The tape of claim 1, wherein the particles are bound to the adhesive.

13. A surgical implant, comprising:
a metal implant component;
a biodegradable tape comprised of a polymer and an antibiotic, the tape being wrapped around at least a portion of the metal component.

14. The implant of claim 13, wherein the antibiotic is in a plurality of groups of spherical particles wherein the particles of each group has the same diameter as other particles in that group with a margin of error of ±20% or less;
wherein each group comprises 100 or more particles and further wherein particles of each group dissolve at a rate different from a rate at which the particles of other groups dissolve.

15. The tape of claim 14,
wherein the plurality of groups comprise a first group of particles have 1,000 square centimeters or more of surface area more than the first group of particles; and
a second group of particles have 2,000 square centimeters or more of surface area more than the first group of particles.

16. The implant of claim 15,
wherein the second group of particles have 5,000 square centimeters or more of surface area more than the first group of particles; and
a third group is present wherein the third group of particles have 10,000 square centimeters or more of surface area more than the second group of particles.

17. The implant of claim 14, wherein each group of spherical particles is bound to an adhesive.

18. The implant of claim 17, wherein the metal component is a surgical screw.

19. The implant of claim 13, wherein the tape is comprised of collagen.

20. The implant of claim 14, wherein the particles of each group dissolve at a rate per unit of time which is different from a rate of dissolution of any other of the groups of particles by an amount of about 10% or more.

21. The immplant of claim 14, wherein the particles of each group dissolve at a rate per unit of time which is different from a rate of dissolution of any other of the groups of particles by an amount of about 25% or more.

22. The tape comprising a biodegradable polymer and an antibiotic, the tape having a length of from 5 mm to 100 meters and a width of from 1 mm to 10 cm.

23. The tape of claim 22, wherein the thickness of the tape is from 0.01 mm to 5 mm.

24. A metal surgical screw with circular indentations in a surface, the indentations having bound thereto groups of particles, comprising:
a first group of spherical coated particles wherein each particle of the first group has an outer diameter substantially the same as other particles in the first group with a margin of error of ±20% or less and wherein the particles have a flowable liquid center surrounded by an outer coating; and
a second group of coated spherical particles wherein each particle of the second group has substantially the same diameter as other particles in the second group with a margin of error of ±20% or less and wherein the coated spherical particles of the second group are comprised of a liquid flowable core surrounded by an outer coating.

25. The surgical screw of claim 24, wherein the flowable liquid center of the spherical particles of the first group and the flowable liquid center of the spherical particles of the second group are comprised of a solution of a pharmaceutically active drug; and
wherein upon administration to a biological system the particles of the first group release the liquid core at a different time from the time at which the particles of the second group release the inner core.

26. The surgical screw of claim 24, further comprising:
a third group of coated spherical particles wherein each particle of the third group has the same diameter as other particles in the third group with a margin of error of ±20% or less and wherein the coated spherical particles of the third group are comprised of a liquid flowable core surrounded by an outer coating;
wherein the flowable liquid center of the spherical particles of the third group are comprised of a solution of a pharmaceutically active drug; and
wherein upon implantation in bone the particles of the third group release the liquid core at a different time from particles of the first and second groups.

27. The surgical screw of claim 24, further comprising:
a plurality of additional groups of coated spherical particles wherein the particles of each additional group have the same diameter as other particles in that group with a margin of error of ±20% or less and wherein the coated spherical particles of each additional group are comprised of a liquid flowable core surrounded by an outer coating; and
wherein the flowable liquid centers of the spherical particles of each additional group are comprised of a solution of a pharmaceutically active drug; and
wherein upon administration to a biological system the particles of each group releases the liquid core at a different time from other groups.

28. The surgical screw of claim 24, wherein an adhesive binds the particles to the surface.

29. The formulation of claim 24, wherein the pharmaceutically active drug is an antibiotic.

30. The device of claim 24, wherein the drug is chosen from an antibiotic, an antifungal and an antiviral compound.

31. The surgical screw of claim 24, wherein the coated spherical particles are produced by a process, comprising the steps of:
forcing a liquid formulation comprising a pharmaceutically active drug through a channel of a first feeding source in a manner which causes a stream of the liquid drug to be expelled from a first exit opening at a first velocity;
forcing a liquid comprising a coating material through a second channel concentrically positioned around the first channel in a manner which causes a stream of the liquid coating material to be expelled from a second exit opening at a velocity which is substantially the same as the first velocity whereby the stream of coating material is concentrically positioned around the stream of liquid drug;
forcing a gas through a pressure chamber surrounding the exit openings of the concentrically positioned first and second channels in a manner which causes the gas to exit the pressure chamber from an exit orifice positioned downstream of the concentrically positioned streams of liquid drug and coating material;
wherein the density of the liquid formulation comprising the pharmaceutically active drug is substantially the same as the density of the liquid comprising the coating material, and the gas focuses the concentrically positioned streams to a stable unified jet which flows out of the chamber exit orifice and breaks up into coated particles of the pharmaceutically active drug coated with the coating material.

32. The surgical screw of claim 31, wherein the stable unified jet comprises a diameter dⱼ at a given point A in the stream characterized by the formula:
$${d}_{j} ≅ {( \frac{8 {ρ}_{1}}{{π}^{2} Δ {P}_{g}} )}^{1 / 4} {Q}^{1 / 2}$$
wherein dⱼ is the diameter of the stable unified jet, □ indicates approximately equally to where an acceptable margin of error is ∀ 10%, ρ₁ is the average density of the liquid of the jet and ΔP_{g} is change in gas pressure of gas surrounding the stream at a given point A and Q is the total flow rate of the stable unified jet.

33. The surgical screw of claim 32, wherein dⱼ is a diameter in a range of about 1 micron to about 1 mm.

34. The surgical screw of claim 32,
wherein the stable unified jet has a length in a range of from about 1 micron to about 50 mm;
wherein the stable unified jet is maintained, at least in part, by tangential viscous stresses exerted by the gas on a surface of the jet in an axial direction of the jet; and
wherein the stable unified jet is further characterized by a slightly parabolic axial velocity profile.

35. The surgical screw of claim 32, wherein the particles of pharmaceutically active drug coated with coating material are characterized by having the same diameter with a deviation in diameter from one particle to another in a range of from about ±3% or less.

36. The surgical screw of claim 35, wherein the deviation in diameter from one particle to another is in a range of from about ±1% or less.

37. The surgical screw of claim 32, wherein a coated particle of the first group has a diameter in a range of about 0.1 micron to about 100 microns and other particles of the first group have the same diameter as the given particle with a deviation of about ∀3% or less; and
wherein ΔP=Pₒ−P₁, the difference in pressure through the chamber exit orifice, is equal to or less than twenty times the surface tension of the liquid comprising the coating material with the gas, divided by the radius of the stable unified jet.

38. The surgical screw of claim 24,
wherein the second group of particles have 1,000 square centimeters or more of surface area more than the first group of particles; and
wherein the third group of particles have 2,000 square centimeters or more of surface area more than the second group of particles.

39. The surgical screw of claim 24,
wherein the second group of particles have 5,000 square centimeters or more of surface area more than the first group of particles; and
wherein the third group of particles have 10,000 square centimeters or more of surface area more than the second group of particles.

40. A method, comprising:
inserting a surgical screw into a bone wherein the surgical screw has bound to its surface a plurality of spherical particles which particles comprise an antimicrobial compound; and
allowing the antimicrobial compound to diffuse into the bone in an area surrounding the surgical screw.

41. A method, comprising:
wrapping a metal surgical implant with a tape comprised of a biodegradable polymer and an antibiotic.

42. The method of claim 41, wherein the implant is a screw.

43. The method of claim 41, further comprising:
inserting the implant into a living patient.

44. The method of claim 43, wherein the tape is comprised of pharmaceutical grade collagen.
